(19) United States
(12) Patent Application Publication
(10) Pub. No.: US 2009/0215812 A1
(43) Pub. Date: Aug. 27, 2009

(54) BIOMARKERS FOR EVALUATING LIKELIHOOD OF TUMOR SENSITIVITY TO AN MTOR INHIBITOR

(75) Inventors: Camille L. Bedrosian, Belmont, MA (US); Timothy P. Clackson, Lexington, MA (US); Victor Rivera, Arlington, MA (US)

Correspondence Address: CHOATE, HALL & STEWART LLP, TWO INTERNATIONAL PLACE, BOSTON, MA, 02110, US

(73) Assignee: ARIAD GENE THERAPEUTICS, INC., Cambridge, MA (US)

(21) Appl. No.: 11/913,964

(22) PCT Filed: May 8, 2006
(86) PCT No.: PCT/US06/17770

§ 371 Date: Jan. 20, 2009

(30) Foreign Application Priority Data
May 13, 2005 (GB) 0509748.0

(51) Int. Cl. A61K 31/436; C12Q 1/00; C12Q 1/68; G01N 33/53

(52) U.S. Cl. 514/291; 435/4; 435/6; 435/7.1

(57) ABSTRACT

The present invention provides compositions and methods for evaluating the likelihood that a tumor is sensitive to an mTOR inhibitor, e.g., rapamycin or a rapamycin analog. The invention provides FKBP proteins as biomarkers for predicting the likelihood that a tumor is sensitive to an mTOR inhibitor. The methods include assessing the expression or activity of an FKBP protein, e.g., FKBP 12, in a subject with a tumor or in a sample derived from a tumor. Additional biomarkers and biomarker combinations are also provided. The invention also provides kits containing, e.g., a validated antibody or ligand for assessing the expression or activity of an FKBP protein.

### CROSS REFERENCE TO RELATED APPLICATIONS

This application claims priority to U.S. provisional application Ser. No. 60/679,435 filed May 9, 2005; Ser. No. 60/679,965 filed May 10, 2005; and Ser. No. 60/680,978 filed May 13, 2005, the entirety of which are hereby incorporated by reference.

### BACKGROUND OF THE INVENTION

Despite decades of research, cancer remains a highly lethal disease. Although notable successes have been achieved in treating some cancers such as childhood leukemia and Hodgkin's lymphoma, options for treating many types of cancer remain far from satisfactory. In many cases, the mainstays of therapy remain surgery, radiation, and conventional chemotherapeutic agents that exhibit nonspecific cytostatic or cytotoxic activity, particularly against rapidly dividing cells. As a result, side effects are a significant issue and limit the maximum tolerated dose and thus the efficacy of these agents.

In recent years improved understanding of the molecular basis of cancer and identification of genetic and biochemical alterations that are found in cancers of various types has led to the development of new classes of therapeutic agents that act on specific molecules. Such agents are often referred to as targeted therapeutic agents. Frequently the targeted molecules are components of biological pathways whose dysregulation in particular cells may play a causative role in the development or progression of cancer.

Although these new and highly specific therapies promise to revolutionize cancer treatment, it has become evident that not all cancers are sensitive to any particular therapeutic agent. The effects of a therapeutic agent that acts on a particular molecule or biological pathway can differ, even among tumors that affect the same organ or fall into similar histopathologic categories. As a result, not all patients exhibit a favorable response to treatment. Patients that fail to respond to a therapeutic agent are needlessly exposed to its potential side effects and may forego the opportunity to be treated with an alternative agent to which they might respond.

mTOR (mammalian target of rapamycin, also known as FKBP-rapamycin associated protein, or FRAP) is a protein kinase that plays a role in regulating a variety of cellular processes including cell growth and proliferation. Inhibitors of mTOR are a promising new class of chemotherapeutic agent, and a variety of mTOR inhibitors are currently in clinical trials for treating tumors of different types. However, as in the case of many other chemotherapeutic agents, the response is variable, with some patients experiencing significant benefit while others fail to exhibit a favorable response. There is a need in the art for methods of evaluating the likelihood that a tumor will be sensitive to an mTOR inhibitor, and there is a need in the art for reagents and diagnostic kits for the practice of such methods.

### SUMMARY OF THE INVENTION

The present invention provides a variety of methods and reagents for evaluating the likelihood that a tumor is sensitive to an mTOR inhibitor. The invention is related in part to the discovery that the expression level of FKBP12 protein varies significantly among tumor cell lines derived from a variety of different tumor types and that the level of FKBP12 expression correlates with the sensitivity of the tumor cell lines to an mTOR inhibitor. In particular, tumor cell lines having lower FKBP12 expression levels exhibited reduced sensitivity to treatment with an mTOR inhibitor relative to tumor cell lines with higher FKBP12 expression. The invention therefore establishes FKBP12 as a biomarker for assessing the likelihood that a tumor is sensitive to an mTOR inhibitor and for assessing the likelihood that a subject suffering from a tumor will respond to treatment with an mTOR inhibitor.

In one aspect, the invention provides a method for evaluating the likelihood that a tumor is sensitive to an mTOR inhibitor comprising a step of assessing the expression level or activity of an FKBP protein in a sample derived from a subject in need of such evaluation, wherein the subject has a tumor, or assessing an indicator of the expression or activity of the FKBP protein in the tumor in vivo, wherein the value of the indicator, relative to a reference value for the indicator, is predictive of the likelihood that the tumor is sensitive to the mTOR inhibitor. In certain embodiments of the invention the FKBP protein is FKBP12.

The invention is also related to the identification of a variety of additional biomarkers that are of use individually, in conjunction with one another, and/or in conjunction with FKBP expression or activity level for assessing the likelihood that a tumor is sensitive to an mTOR inhibitor and for assessing the likelihood that a subject suffering from a tumor will respond to treatment with an mTOR inhibitor. In certain embodiments the method for evaluating the likelihood that a tumor is sensitive to an mTOR inhibitor comprises the further step of assessing at least one additional indicator of the likelihood that the tumor is sensitive to an mTOR inhibitor, wherein the value of the indicator, relative to a reference value for the indicator, is predictive of the likelihood that the tumor is sensitive to the mTOR inhibitor. The at least one additional indicator is preferably selected from the group consisting of: (a) the proportion or level of TSC2 protein that is phosphorylated; (b) the proportion or level of AKT protein that is phosphorylated; (c) the proportion or level of S6 protein that is phosphorylated; (d) the proportion or level of S6 kinase protein that is phosphorylated; (e) the proportion or level of 4E-BP1 protein that is phosphorylated; (f) the proportion or level of mTOR protein that is phosphorylated; (g) the level of cyclin D1 mRNA or protein, cyclin D3 mRNA or protein, myc mRNA or protein, or any combination of these (h) the level of HIF-1α mRNA or protein, HIF-2α mRNA or protein; HIF-1β mRNA or protein, or any combination of the foregoing; (i) the level of VHL mRNA or protein or a mutation affecting VHL expression or activity; (j) the level of RHEB mRNA or protein or the activity of RHEB protein; (k) the level of eIF4E mRNA or protein; (l) the level of p27Kip1 mRNA or protein; (m) the level of VEGF-R mRNA or protein; (n) the level of IGF-1R mRNA or protein; (o) the level of PTEN mRNA, protein, or activity, or presence of a mutation affecting PTEN expression or activity; (p) the level of Raptor mRNA, protein, or activity, or presence of a mutation affecting Raptor expression or activity; (q) the level of GβL mRNA, protein, or activity, or presence of a mutation affecting GβL expression or activity; (r) the level of a circulating VEGF polypeptide; and (s) the level of circulating endothelial cells (CECs), the level of circulating endothelial progenitor cells (CEPs), or both. In certain embodiments the additional indicator is obtained by performing one or more functional imaging studies. In certain embodiments of the invention one or more indicators is assessed prior to and following administration of an mTOR inhibitor, and an alteration in the value of the indicator is indicative of the likelihood that the tumor is sensitive to an mTOR inhibitor.

In another aspect, the invention provides a method of evaluating the likelihood that a subject with a tumor will exhibit a favorable response to treatment with an mTOR inhibitor. The method comprises the step of evaluating the likelihood that the tumor is sensitive to an mTOR inhibitor by assessing the expression level or activity of an FKBP protein in a sample derived from the tumor, or assessing an indicator of the expression or activity of the FKBP protein in the tumor in vivo, wherein the value of the indicator, relative to a reference value for the indicator, is predictive of the likelihood that the tumor is sensitive to the mTOR inhibitor. In certain embodiments of the invention the FKBP protein is FKBP12. In certain embodiments the method comprises the further step assessing at least one additional indicator of the likelihood that the tumor is sensitive to an mTOR inhibitor, wherein the value of the indicator, relative to a reference value for the indicator, is predictive of the likelihood that the tumor is sensitive to the mTOR inhibitor.

In another aspect, the invention provides a method of selecting a subject for treatment with an mTOR inhibitor, wherein the subject has a tumor. The method comprises the step of evaluating the likelihood that the tumor is sensitive to an mTOR inhibitor by assessing any of a variety of indicators, and/or evaluating the likelihood that the subject will exhibit a favorable response to the mTOR inhibitor by assessing any of a variety of indicators.

In another aspect, the invention provides a method of selecting an appropriate therapeutic agent for a subject with a tumor.

The invention further provides a method for treating a subject suffering from a tumor, the method comprising the steps of: (a) evaluating the likelihood that the subject will exhibit a favorable response to an mTOR inhibitor by a method that comprises evaluating the likelihood that the tumor is sensitive to an mTOR inhibitor by assessing one or more of a variety of indicators and (b) administering an mTOR inhibitor to the subject.

In another aspect, the invention provides a variety of kits. For example, the invention provides a kit for evaluating the likelihood that a tumor is sensitive to an mTOR inhibitor, the kit comprising a reagent for assessing an indicator of the expression or activity of an FKBP protein in a sample obtained from a subject with a tumor, wherein the reagent has been demonstrated to be of use in evaluating the likelihood that a tumor is sensitive to an mTOR inhibitor. The invention further provides a kit comprising a reagent that specifically binds to an FKBP protein, e.g., FKBP12 and (b) an additional item selected from the group consisting of: (a) an antibody that specifically binds to a protein selected from the group consisting of: mTOR, PTEN, Akt, Raptor, GβL, or any other component of a pathway involving mTOR. In certain embodiments at least one of the antibodies is specific for a phosphorylated form of the protein.

The invention further provides automated staining instruments of use in evaluating the likelihood that a tumor is sensitive to an mTOR inhibitor. The invention further provides computer-readable medium on which is stored information related to samples, tumors, and/or subjects, the value of indicators in said samples, tumors, and/or subjects, and, optionally, the sensitivity or responsiveness of such tumors and/or subjects to administration of an mTOR inhibitor.

Unless otherwise stated, the invention makes use of standard methods of molecular biology, cell culture, flow cytometry, histology, immunology, immunohistochemistry, etc. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. The materials, methods, and examples are not intended to be limiting.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. Where ranges are given, endpoints are included.

This application refers to various patents and other publications. The contents of all of these are incorporated by reference. In addition, the following publications are incorporated herein by reference: Ausubel, F. (ed.) Current Protocols in Molecular Biology, Current Protocols in Immunology, Current Protocols in Protein Science, and Current Protocols in Cell Biology, all John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Harlow, E., and Lane, D., Antibodies: A Laboratory Manual, Cold Spring Harbor Laboratory Press, Cold Spring Harbor, 1988; Goodman and Gilman's The Pharmacological Basis of Therapeutics, 10^{th} Ed. McGraw Hill, 2001, and Katzung, B. (ed.) Basic and Clinical Pharmacology, McGraw-Hill/Appleton & Lange; 8th edition (Sep. 21, 2000), and Devita, V., et al. (eds.), Cancer: Principles And Practice Of Oncology, 7^{th} ed., Lippincott Williams & Wilkins, 2004. In the event of a conflict between any of the incorporated references and the instant specification, the specification shall control.

### BRIEF DESCRIPTION OF THE DRAWING

FIG. 1 shows a Western blot of FKBP12 protein expression in a panel of tumor cell lines.

### DEFINITIONS

“Antibody”, as used herein, refers to an immunoglobulin that binds to an antigen. An antibody may be natural or wholly or partially synthetically produced. An antibody may be derived from natural sources, e.g., purified from an animal such as a rodent, rabbit, or chicken, that has been immunized with an antigen or a construct that encodes the antigen. An antibody may be a member of any immunoglobulin class, including any of the human classes: IgG, IgM, IgA, IgD, and IgE. The antibody may be an antibody fragment such as an Fab′, F(ab′)₂, scFv (single-chain variable) or other fragment that retains an antigen binding site, or a recombinantly produced scFv fragment, including recombinantly produced fragments that comprise an immunoglobulin antigen binding domain. See, e.g., Allen, T., Nature Reviews Cancer, Vol. 2, 750-765, 2002, and references therein. Antibodies, antibody fragments, and/or protein domains comprising an antigen binding site may be generated and/or selected in vitro, e.g., using techniques such as phage display (Winter, G. et al. 1994. Annu. Rev. Immunol. 12:433-455, 1994), ribosome display (Hanes, J., and Pluckthun, A. Proc. Natl. Acad. Sci. USA. 94:4937-4942, 1997), etc. An antibody may be a “humanized” antibody in which for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes.

An antibody may be polyclonal (e.g., an affinity-purified polyclonal antibody) or monoclonal.

The terms “approximately” or “about” in reference to a number are generally include numbers that fall within a range of 5% in either direction (greater than or less than) of the number unless otherwise stated or otherwise evident from the context (except where such number would exceed 100% of a possible value).

The terms “assess”, “assessing”, and the like are understood broadly and include obtaining information, e.g., determining a value, whether through direct examination or by receiving information from another party that performs the examination.

The term “biomarker” is used herein as understood in the art. The term can refer to an indicator that provides information about a process, phenotype, or outcome of interest, e.g., the sensitivity of a tumor to a chemotherapeutic agent, or the response of a subject to a chemotherapeutic agent. In general, the value of such an indicator is correlated with the process, phenotype, or outcome of interest. The term “biomarker” can also refer to a molecule that is the subject of an assay or measurement the result of which provides information about a process, phenotype, or outcome of interest. For example, an elevated expression level of a particular protein can be an indicator that a subject has a disease. The expression level of the protein, an elevated expression level of the protein, and the protein itself can all be referred to as “biomarkers”.

An “effective amount” of an active agent refers to the amount of the active agent sufficient to elicit a desired biological response, e.g., to treat or prevent a disease or condition in a subject. As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses. In the case of a tumor, an effective amount may reduce the growth rate of the tumor (e.g., reduce the proliferation rate of tumor cells), reduce or prevent local spread of the tumor, reduce or prevent invasion of other organs, reduce or prevent metastasis, alleviate one or more symptoms associated with the tumor, etc. An effective amount of a therapeutic agent for a tumor may kill tumor cells (i.e., may be cytotoxic) or may prevent their further proliferation (i.e., may be cytostatic). An effective amount of a therapeutic agent may be an amount sufficient to elicit a partial or complete response, cause stabilization of disease, increase time to progression, etc.

“Expression” refers to the process by which a polynucleotide is transcribed to produce mRNA (mRNA expression) and by which the mRNA is translated to produce a polypeptide (protein expression). Expression may include processing, such as splicing of an mRNA.

The term “gene”, as used herein, has its meaning as understood in the art. In general, a gene is understood to include gene regulatory sequences (e.g., promoters, enhancers, etc.) and/or intron sequences and typically also includes a sequence that encodes a polypeptide. It will be appreciated that a “gene” can refer to a nucleic acid that does not encode a protein but rather encodes a functional RNA molecule such as an rRNA, short hairpin RNA (shRNA), tRNA, etc.

A “gene product” or “expression product” is an RNA transcribed from the gene (e.g., either pre- or post-processing) or a polypeptide encoded by an RNA transcribed from the gene (e.g., either pre- or post-modification).

The term “hybridize” refers to the interaction between two or more nucleic acid segments comprising or consisting of complementary portions such that a complex stabilized by hydrogen bonds is formed. Typically a first and second nucleic acid segment will hybridize to each other if the Tm of a duplex formed by the first and second nucleic acid segments is less than 15° C. below, preferably less than 10° C. below the Tm of a duplex that would be formed by the second nucleic acid and a third nucleic acid that is the same length as, and 100% complementary to, the second nucleic acid and contains nucleosides and internucleosidic linkages of the same type. Hybridization conditions suitable for various applications are known in the art and are found in standard reference works, e.g., Ausubel, supra, and Sambrook, supra. Hybridization reactions can be performed under conditions of different “stringency”. The stringency of a hybridization reaction relates to the degree of complementarity necessary for hybridization to occur or be maintained, with low stringency conditions being those in which a lesser degree of complementary is sufficient and high stringency conditions being those in which a higher degree of complementarity is needed. In an exemplary embodiment, stringent hybridization conditions comprise 6 X sodium chloride/sodium citrate (SSC) and 0.1% SDS at a temperature 10-15° C. below the Tm of a perfectly complementary duplex, followed by washing 1-2 times for 30 minutes in 2×SSC and 0.1% SDS at a temperature 25° C. below the Tm of a perfectly complementary duplex. The Tm is defined as the temperature at which 50% of a nucleic acid and its perfect complement are in duplex in solution. Methods for calculating or experimentally determining Tm are known in the art.

“Imaging”, “imaging study”, “imaging procedure” and like terms are used consistently with their meaning in the art to refer to a process that includes producing one or more “pictures” or representations of one or more body structures or tissues (in which blood is considered a tissue), typically one or more internal body structures or tissues, including an abnormal growth such as a tumor, a foreign body, etc., that may exist within such body structure or tissue. A wide variety of imaging procedures are known in the art and are commonly used for diagnostic and/or therapeutic purposes. The image may reflect any of a variety of properties and/or features of the tissue or body structure including physical, anatomical, biochemical, and/or metabolic features. Examples of imaging modalities include, but are not limited to, radiographic (e.g., X-rays, computed tomography (CT), magnetic resonance imaging (MRI), positron emission tomography (PET), ultrasound, scintigraphy). Some imaging methods involve injection of small amounts of radioactively labeled (or otherwise detectably labeled) substances (e.g., radio-isotopes) into a subject. The radioactive substances may concentrate directly in an organ/tissue or may be attached to a targeting agent such as an antibody. An image produced by an imaging procedure may be analysed and/or interpreted visually and/or using automated image analysis.

The term “indicator” is used broadly herein to refer to any parameter, attribute, or characteristic of a molecule, sample, or subject that can be detected, determined, and/or quantitated to obtain a value that can be compared with another value, e.g., a reference value. A value can be qualitative or quantitative and can be a range of values. An indicator can be, for example, the level of expression or activity of a protein, the proportion of a protein that is phosphorylated, the presence or number of cells of a particular type, presence or absence of a mutation, a parameter obtained from an imaging study, etc.

The term “inhibitor” is used herein to refer to any molecule or other agent capable of inhibiting (e.g., partially or completely blocking, retarding, interfering with) one or more biological activities (e.g., a physiologically significant enzymatic activity) of a target molecule such as mTOR. Examples include small molecules such as rapamycin and rapamycin analogs, antibodies, short interfering RNA (siRNA), short hairpin RNA (shRNA), antisense molecules, ribozymes, etc. An inhibitor may inhibit synthesis of a target polypeptide (e.g., by inhibiting synthesis of, or causing destabilization of, an mRNA that encodes the polypeptide, or by inhibiting translation of the polypeptide), may accelerate degradation of the polypeptide, may inhibit activation of the polypeptide (e.g., by inhibiting an activating modification such as phosphorylation or cleavage), may block an active site of the polypeptide, may cause a conformational change in the polypeptide that reduces its activity, may cause dissociation of an active complex containing the polypeptide, etc. An inhibitor may act directly by physical interaction with a target molecule, or indirectly, for example by interacting with a second molecule whose activity contributes to activation of the target molecule (e.g., a molecule that activates the target molecule, e.g., by phosphorylating it), by competing with the target molecule for binding to a substrate, activator, or binding partner needed for activity of the target molecule, etc.

The term “isolated” means 1) separated from at least some of the components with which it is usually associated in nature; 2) prepared or purified by a process that involves the hand of man; and/or 3) not occurring in nature.

“Operably linked” or “operably associated” refers to a relationship between two nucleic acid sequences wherein the expression of one of the nucleic acid sequences is controlled by, regulated by, modulated by, etc., the other nucleic acid sequences, or a relationship between two polypeptides wherein the expression of one of the polypeptides is controlled by, regulated by, modulated by, etc., the other polypeptide. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport, stability, or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence, or a polypeptide that is operatively linked to a second polypeptide, is covalently linked, either directly or indirectly, to such a sequence, although any effective three-dimensional association is acceptable.

“Polynucleotide” or “oligonucleotide” refers to a polymer of nucleotides, typically comprising at least three nucleotides. As used herein, an oligonucleotide is typically less than 100 nucleotides in length. A polynucleotide or oligonucleotide may also be referred to as a nucleic acid. Naturally occurring nucleic acids include DNA and RNA. A nucleotide comprises a nitrogenous base, a sugar molecule, and a phosphate group. A nucleoside comprises a nitrogenous base linked to a sugar molecule. As used herein, the term includes natural nucleosides in their 2′-deoxy and 2′-hydroxyl forms as described in Kornberg and Baker, DNA Replication, 2nd Ed. (Freeman, San Francisco, 1992) and nucleoside analogs. For example, natural nucleosides include adenosine, thymidine, guanosine, cytidine, uridine, deoxyadenosine, deoxythymidine, deoxyguanosine, and deoxycytidine. Nucleoside “analogs” refers to synthetic nucleosides having modified base moieties and/or modified sugar moieties, e.g. described generally by Scheit, Nucleotide Analogs (John Wiley, New York, 1980). Such analogs include synthetic nucleosides designed to enhance binding properties, reduce degeneracy, increase specificity, and the like. In naturally occurring nucleic acids (DNA or RNA), adjacent nucleosides are linked 3′ to 5′ phosphodiester bond. However, polynucleotides and oligonucleotides containing modified backbones or non-naturally occurring internucleoside linkages can also be used in the present invention. Examples include, but are not limited to, phosphorothioate and 5′-N-phosphoramidite linkages. See U.S. Pub. No. 20040092470 and references therein for further discussion of various nucleotides, nucleosides, and backbone structures and methods for producing them.

A polynucleotide may be of any size or sequence and may be single- or double-stranded. Polynucleotides in the form of DNA, cDNA, genomic DNA, RNA, mRNA and synthetic DNA or RNA within the scope of the present invention. DNA may be double-stranded or single-stranded, and if single-stranded may be a coding (sense) strand or non-coding (anti-sense) strand. A polynucleotide may be, for example, a modified or unmodified circular plasmid, a linearized plasmid, a cosmid, a modified or unmodified viral genome, a gene or gene fragment, messenger RNA, a short interfering RNA (siRNA) or short hairpin RNA (shRNA), an antisense oligonucleotide, a ribozyme, etc. In certain embodiments the polynucleotide has been engineered using recombinant techniques (for a more detailed description of these techniques, please see Ausubel et al., Current Protocols in Molecular Biology, supra, and Molecular Cloning: A Laboratory Manual, supra. A polynucleotide may be obtained from natural sources and purified from contaminating components found normally in nature. A polynucleotide may be synthesized using enzymatic techniques, either within cells or in vitro. The polynucleotide may also be chemically synthesized in a laboratory. In a preferred embodiment, the polynucleotide is synthesized using standard solid phase chemistry. The polynucleotide may be modified by chemical or biological means. In certain preferred embodiments, these modifications lead to increased stability of the polynucleotide. Modifications include methylation, phosphorylation, end-capping, etc. A polynucleotide may be labeled, e.g., by incorporation or attachment of one or more detectable labels.

“Polypeptide”, as used herein, refers to a polymer of amino acids. A protein is composed of one or more polypeptides. A peptide is a relatively short polypeptide, typically between about 2 and 60 amino acids in length. Peptide, polypeptide, or protein, may refer to an individual peptide, polypeptide, or protein, or a collection thereof.

Polypeptides used herein preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Preventing” refers to causing a disease, disorder, condition, or symptom or manifestation of such, or worsening of the severity of such, not to occur.

“Purified”, as used herein, means separated from many other compounds or entities. A compound or entity may be partially purified, substantially purified, or pure. A compound or entity is considered pure when it is removed from substantially all other compounds or entities, i.e., is preferably at least about 90%, more preferably at least about 91%, 92%, 93%, 94%, 95%, 96%, 97%, 98%, 99%, or greater than 99% pure. A partially or substantially purified compound or entity may be removed from at least 50%, at least 60%, at least 70%, or at least 80% of the material with which it is naturally found, e.g., cellular material such as cellular proteins and/or nucleic acids.

The term “primer” refers to an oligonucleotide, whether natural or synthetic, that hybridizes to a nucleic acid “target” or “template” present in a sample of interest and is capable of acting as a point of initiation of nucleic acid synthesis under conditions in which primer extension, e.g., polymerase-catalyzed primer extension can occur. The appropriate length of a primer depends on the intended use of the primer, but typically ranges from about 15 to about 35 nt. In some cases a primer may be longer, e.g., up to about 60 nt in length. Short primer molecules generally require cooler temperatures to form sufficiently stable hybrid complexes with a template. A primer need not be 100% complementary to the template but must be sufficiently complementary to hybridize with the template for primer elongation to occur. The primer can be extended by one or more nucleotides, depending on the particular application. Primers are typically used for replication of a nucleic acid (amplification) and/or for detecting a mutation by any of a variety of methods. A primer can also be used for a variety of purposes that do not require polymerase-catalyzed polymerization. For example, a primer can be used in a reaction such as ligation. A primer can also be used as a probe for detection of a complementary nucleic acid. Methods for selecting primers, e.g., using the Tm value, are well known in the art and computer programs for doing so are widely available.

The term “probe”, when referring to a nucleic acid, refers to a nucleic acid that can hybridize with and thereby detect the presence of a complementary nucleic acid The probe should be sufficiently complementary to the nucleic acid being detected so that specific hybridization can occur under the hybridization stringency conditions used. The probe may comprise a label or means for attachment of a label. Suitable labels include, but are not limited to radioisotopes, fluorescent moieties, chemiluminescent compounds, dyes, and proteins, including enzymes.

The term “reference value” is used broadly herein to refer to a value for any parameter, attribute, or characteristic of a molecule, sample, or subject that can be detected, determined, and/or quantitated and can be compared with another value, e.g., a value obtained for that parameter, attribute, or characteristic from a molecule, sample, or subject of interest. A reference value can be qualitative or quantitative and can be a range of values. A reference value can be, for example, the level of expression or activity of a protein, the proportion of a protein that is phosphorylated, the presence or number of cells of a particular type, presence or absence of a mutation, a parameter obtained from an imaging study, etc. Typically a reference value is obtained from a population of molecules, samples, or subjects (reference population) known to exhibit one or more characteristics or features, and a comparison of the reference value for a particular parameter, characteristic or attribute with and a value for that parameter, characteristic, or attribute obtained from a molecule, sample, or subject of interest provides information regarding the similarity between the reference population and the molecule, sample, or subject of interest with respect to those characteristics or features.

The term “regulatory element” or “regulatory sequence” in reference to a nucleic acid is generally used herein to describe a portion of nucleic acid that directs or increases one or more steps in the expression (particularly transcription, but in some cases other events such as splicing or other processing) of nucleic acid sequence(s) with which it is operatively linked. The term includes promoters and can also refer to enhancers and other transcriptional control elements. Promoters are regions of nucleic acid that include a site to which RNA polymerase binds before initiating transcription and that are typically necessary for even basal levels of transcription to occur. Generally such elements comprise a TATA box. Enhancers are regions of nucleic acid that encompass binding sites for protein(s) that elevate transcriptional activity of a nearby or distantly located promoter, typically above some basal level of expression that would exist in the absence of the enhancer. Regulatory sequences may direct constitutive expression of a nucleotide sequence (e.g., expression in most or all cell types under typical physiological conditions in culture or in an organism) or may direct cell or tissue-specific and/or inducible expression. Regulatory elements may also inhibit or decrease expression of an operatively linked nucleic acid.

A “response” is a change in a tumor or subject relative to a previous state. A response to a therapeutic agent occurs following, and related to, administration of one or more doses of the agent. A “favorable response” as used herein, refers to any biological, chemical, or physical response that is recognized by those skilled in the art as being beneficial to a subject, e.g., as indicating a decreased rate of tumor growth or progression. A favorable response can be a partial or complete response, stabilization of disease, increased time to progression, etc., or any therapeutically beneficial change in the tumor or subject, relative to the condition of the tumor or subject that would exist in the absence of treatment. One of ordinary skill in the art will appreciate that a variety of criteria are available to determine whether a tumor or subject is exhibiting or has exhibited a favorable response. Such criteria include clinical criteria, imaging criteria, criteria based on tissue biopsy, etc. Standardized criteria can be used and may be preferred for clinical trials. For example, the Response Evaluation Criteria in Solid Tumors (RECIST) criteria (Therasse, P., et al, J. Natl. Cancer Inst., 92:205-16, 2000, and updated versions thereof) can be used. Additional criteria include, but are not limited to, inhibition of cancer cell growth, proliferation and/or survival; tumor shrinkage; etc. Generally if a tumor is said to exhibit a favorable response to a therapeutic agent, then a subject suffering from the tumor is said to exhibit a favorable response, and vice versa. It will be appreciated that evaluating the likelihood that a tumor or subject will exhibit a favorable response is equivalent to evaluating the likelihood that the tumor or subject will not exhibit favorable response, i.e., will exhibit a lack of response or be “non-responsive”.

The term “sample” refers to any specimen that contains cellular material, e.g., contains a cell or collection of cells or a cell lysate. A sample is typically obtained from a subject. The sample can be a body fluid such as blood, lymph, ascites fluid, cerebrospinal fluid, urine; a washing or lavage such as a ductal lavage or bronchioalveolar lavage; an aspirate such as a fine needle aspirate; a scraping; a bone marrow specimen; a tissue biopsy specimen; a surgical specimen, etc. Preferably the sample, when obtained, contains intact cells. The cells typically originate from a tumor and may be isolated directly from the tumor or may be circulating in the blood. In certain embodiments the cells originate from blood vessels that supply a tumor. In certain embodiments the sample contains circulating endothelial cells, circulating endothelial progenitor cells, or both. The sample, or a portion thereof, may be subjected to any of a variety of processing steps and is still considered to be a sample derived from the subject.

A tumor or tumor cell line is “sensitive” to a therapeutic agent if the agent inhibits (i.e., reduces) the growth rate of the tumor or tumor cell line. Typically the growth rate of the tumor or tumor cell line is detectably lower following exposure to the agent and/or in the presence of the agent (e.g., after administration of the agent to a subject, addition of the agent to tissue culture medium, etc.) than it was prior to the exposure and/or in the absence of the agent. Preferably the growth rate, e.g., cell proliferation rate, is decreased by at least a predetermined amount. For example, in certain embodiments a cell line or tumor is considered sensitive to an agent if the proliferation rate following exposure to the agent is reduced by at least 10%, at least 20%, at least 30%, at least 40%, at least 50%, at least 60%, at least 70%, at least 80%, at least 90%, at least 100%, at least 150% (1.5 fold), at least 200% (2-fold), at least 3-fold, at least 5-fold, at least 10-fold, at least 20-fold, or more, relative to the growth rate prior to exposure to the agent. In some embodiments the proliferation rate is reduced to 0, or the number of cells decreases. For example, the number of cells may decline at a rate that is at least 10%, at least 20%, at least 30%, at least 40%, at least 50%, at least 60%, at least 70%, at least 80%, at least 90%, at least 100%, at least 150% (1.5 fold), at least 200% (2-fold), at least 3-fold, at least 5-fold, at least 10-fold, or at least 20-fold, as great as the proliferation rate prior to exposure to the agent. A predetermined amount may be any other value that falls within any subrange, as has any specific value (specified to the tenths place), within the limits of the values set forth above.

It will be appreciated that the exposure can be a single exposure or can be ongoing exposure, e.g., as when a patient is administered a course of a chemotherapeutic agent that includes administration of multiple doses over a period of time. Growth typically refers to cell proliferation. In the case of a tumor, cell proliferation typically results in an increase in volume of the tumor. A tumor or tumor cell line that is sensitive to a therapeutic agent is said to “respond” to the agent. A tumor or tumor cell line that is not sensitive to a therapeutic agent is said to be “resistant” or “non-responsive” to the agent.

A variety of methods can be used to measure the ability of an agent to inhibit cell proliferation. Typical in vitro assays measure the metabolic rate of the cells or the synthesis of macromolecules such as DNA. Measuring incorporation of radiolabeled thymidine into DNA (e.g., using autoradiography or flow cytometry) has long been considered the gold standard for measuring DNA synthesis. Commercially available assays for measuring cell proliferation include the MTT, XTT, or WST-1 assays (Roche Applied Science; Promega), which are based on the conversion of colored tetrazolium salts into compounds of different colors by metabolically active cells. Of course cell counting, either using visual methods or using automated methods such as Coulter counting, cytometry, etc., can also be used. In vivo methods for assessing tumor sensitivity to an agent include a variety of imaging techniques described elsewhere herein. It may be of interest to determine an “IC50” or “IC25” for an agent, which refers to the concentration that reduces cell number by 50% or 25%, respectively.

“Small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

“Specific binding” generally refers to a physical association between a target polypeptide (or, more generally, a target molecule) and a binding molecule such as an antibody or ligand. The association is typically dependent upon the presence of a particular structural feature of the target such as an antigenic determinant or epitope recognized by the binding molecule. For example, if an antibody is specific for epitope A, the presence of a polypeptide containing epitope A or the presence of free unlabeled A in a reaction containing both free labeled A and the binding molecule that binds thereto, will reduce the amount of labeled A that binds to the binding molecule. It is to be understood that specificity need not be absolute but generally refers to the context in which the binding occurs. For example, it is well known in the art that numerous antibodies cross-react with other epitopes in addition to those present in the target molecule. Such cross-reactivity may be acceptable depending upon the application for which the antibody is to be used. One of ordinary skill in the art will be able to select antibodies or ligands having a sufficient degree of specificity to perform appropriately in any given application (e.g., for detection of a target molecule, for therapeutic purposes, etc). It is also to be understood that specificity may be evaluated in the context of additional factors such as the affinity of the binding molecule for the target versus the affinity of the binding molecule for other targets, e.g., competitors. If a binding molecule exhibits a high affinity for a target molecule that it is desired to detect and low affinity for nontarget molecules, the antibody will likely be an acceptable reagent. Once the specificity of a binding molecule is established in one or more contexts, it may be employed in other, preferably similar, contexts without necessarily re-evaluating its specificity. Binding of two or more molecules with one another may be considered specific if the affinity (equilibrium dissociation constant, Kd) is at least 10⁻³ M, preferably 10⁻⁴ M, more preferably 10⁻⁵ M, e.g., 10⁻⁶ M, 10⁻⁷ M, 10⁻⁸ M, or 10⁻⁹ M under the conditions tested.

“Subject”, as used herein, refers to an individual who is a candidate for administration of an mTOR inhibitor or to whom an mTOR inhibitor is or has been administered. The subject may have or be at risk of developing a tumor. A “normal subject” is an individual not known to have or to be at elevated risk for developing a tumor. Preferred subjects are mammals, particularly humans. Other subjects include domesticated mammals (e.g., dogs, cats, etc.) and non-human primates.

“Treating”, as used herein, can generally include reversing, alleviating, inhibiting the progression of, or reducing the likelihood of the disease, disorder, or condition to which such term applies, or one or more symptoms or manifestations of such disease, disorder or condition.

“Tumor” refers to an abnormal mass of tissue that results from excessive cell division. A tumor can be benign (not cancerous) or malignant (cancerous). “Tumor” includes disorders characterized by excessive division of hematopoietic cells. Such disorders include malignant and premalignant hematologic disorders such as leukemia, lymphoma, myeloma, and myeloproliferative disorders. Tumors can be diagnosed using any of a variety of art-accepted methods including physical diagnosis, imaging studies, histopathology (e.g., performed on a cell or tissue sample), biochemical tests, etc. Specific, non-limiting examples of tumors include sarcomas, prostate cancer, breast cancer, endometrial cancer, hematologic tumors (e.g., leukemia, Hodgkin's and non-Hodgkin's lymphoma, multiple myeloma and other plasma cell disorders, myeloproliferative disorders), brain tumors (e.g., low grade astrocytoma, anaplastic astrocytoma, glioblastoma multiforme, oligodendroglioma, and ependymoma), and gastrointestinal stromal tumors (GIST). Sarcomas include osteosarcoma, Ewing's sarcoma, soft tissue sarcoma, and leiomyosarcoma. Additional examples of malignant tumors include small cell and non-small cell lung cancer, kidney cancer (e.g., renal cell carcinoma), hepatocellular carcinoma, pancreatic cancer, esophageal cancer, colon cancer, rectal cancer, stomach cancer, breast cancer, ovarian cancer, bladder cancer, testicular cancer, thyroid cancer, head and neck cancer, thyroid cancer, etc.

“Vector”, as used herein, refers to a nucleic acid molecule capable of mediating entry of, e.g., transferring, transporting, etc., a second nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids (typically DNA molecules although RNA plasmids are also known), cosmids, and viral vectors. As is well known in the art, the term “viral vector” may refer either to a nucleic acid molecule (e.g., a plasmid) that includes virus-derived nucleic acid elements that typically facilitate transfer or integration of the nucleic acid molecule (examples include retroviral or lentiviral vectors) or to a virus or viral particle that mediates nucleic acid transfer (examples include retroviruses or lentiviruses). As will be evident to one of ordinary skill in the art, viral vectors may include various viral components in addition to nucleic acid(s).

Expression vectors are vectors that include regulatory sequence(s), e.g., expression control sequences (e.g., a promoter and/or other expression signals and, optionally, 3′ sequences, such as 3′ regulatory sequences or termination signals), sufficient to direct transcription of an operably linked nucleic acid segment. The nucleic acid segment may, but need not be, a protein coding sequence. The nucleic acid segment may be chimeric, meaning that it includes more than one sequence of distinct origin that are fused together by recombinant DNA techniques, resulting in a nucleotide sequence that does not occur naturally. The term “expression vector” can refer to a vector either before or after insertion of the operably linked nucleic acid segment. An expression vector comprises sufficient cis-acting elements for expression; other elements for expression can be supplied by the host cell or by an in vitro expression system. Such vectors typically include one or more appropriately positioned sites for restriction enzymes, e.g., to facilitate introduction of the nucleic acid segment to be expressed into the vector.

### DETAILED DESCRIPTION OF CERTAIN EMBODIMENTS OF THE INVENTION

### I. FKBP Proteins as Biomarkers

The present invention relates to the identification of new biomarkers and biomarker combinations that are useful for predicting the likelihood that a tumor is sensitive to an mTOR inhibitor. mTOR is a serine/threonine kinase that has emerged as a central regulator of a number of important cellular processes. mTOR signaling is regulated by several upstream pathways that include both positive and negative regulators, and signaling through mTOR regulates a number of downstream pathways that affect cell size and proliferation. In particular, mTOR phosphorylates a variety of proteins involved in translation initiation, resulting in increased cap-dependent translation and increased translation of ribosomal proteins. Activation of mTOR may result in increased expression of various proteins involved in cell cycle progression. These features, among others, have made mTOR an attractive target for cancer therapy. Several mTOR inhibitors have demonstrated promising results in a variety of tumor types (Sawyers, C. Cancer Cell, 4:343-348, 2003). However, the response has been variable even among tumors that are classified into similar types on clinical and/or histopathologic grounds, and it has heretofore not been possible to predict which tumors are likely to be sensitive.

The present invention provides methods and reagents for evaluating the likelihood that a tumor is sensitive to an mTOR inhibitor, e.g., a rapamycin analog. The invention is based in part on the discovery of a correlation between the expression level of an FK506 binding protein (FKBP protein) in a panel of human tumor cell lines and the likelihood that the tumor cell lines are sensitive to an mTOR inhibitor. FKBPs are the cytosolic receptors for macrolides such as FK506, FK520, rapamycin, and rapamycin analogs, and are highly conserved across species lines. When bound to these ligands, FKBPs bind to and inhibit a variety of cellular proteins. In particular, rapamycin binds to FKBP12, and the FKBP12-rapamycin complex binds to mTOR, thereby inhibiting mTOR activity. Rapamycin analogs such as temsirolimus (CCI-779), everolimus (RAD001; Certican), and AP23573 behave in a similar manner.

The inventors treated a variety of different tumor cell lines with the mTOR inhibitor AP23573, a rapamycin analog that has shown promise as a cancer chemotherapeutic agent in a number of clinical trials, determined the sensitivity of these cell lines to the agent, and measured their FKBP12 expression level. FKBP12 is generally considered to be ubiquitously expressed at high levels, with particularly high expression in the brain (Snyder, S., et al., Neuron, 21:283-294). The tumors displayed an unexpected degree of heterogeneity with respect to the expression of FKBP12. Certain tumor cell lines displayed high expression while others displayed low or close to undetectable expression. The inventors observed that tumors having higher FKBP12 expression levels tended to display significant sensitivity to AP23573 (Example 1). Tumors with lower FKBP12 expression were relatively less sensitive to AP23573. This discovery provides the basis for use of FKBP expression or activity levels as biomarkers for evaluating the likelihood that a tumor is sensitive to an mTOR inhibitor and for evaluating the likelihood that a tumor or subject will exhibit a favorable response to treatment with an mTOR inhibitor. The fact that expression of FKBP12 was significantly reduced in cell lines from tumors of a variety of types suggests that these biomarkers will have broad utility for evaluating the likelihood of tumor sensitivity and/or patient response to an mTOR inhibitor. The term “evaluating” is intended to mean that the method provides information useful in forming a judgement as to the likelihood that a tumor is sensitive to an mTOR inhibitor and/or that a subject suffering from a tumor is responding or will respond to treatment with an mTOR inhibitor.

The methods may be used to identify subjects who are likely to respond to an mTOR inhibitor, relative to the likelihood of response in the overall population of subjects. It will be appreciated that the methods do not necessarily predict with complete accuracy whether any particular tumor or subject will exhibit a favorable response but rather indicate that tumors or subjects having certain features are more likely or less likely to exhibit a favorable response than tumors or subjects not having such features. While FKBP12 is exemplified herein, the invention encompasses similar use of the expression level or activity of related FKBP proteins, e.g., FKBP proteins that bind to rapamycin or a rapamycin analog and, when so bound, inhibit the mTOR kinase.

In certain embodiments of the invention expression or activity of an FKBP protein is measured in a sample derived from a tumor (referred to as a “test sample”). In other embodiments of the invention expression or activity of an FKBP protein in a tumor is measured in vivo, e.g., using a functional imaging method.

An expression or activity level can be qualitative or quantitative. Thus a determination of whether a polynucleotide or polypeptide is present or absent (e.g., detectable or undetectable) constitutes determining its expression level in various embodiments of the invention while in other embodiments a quantitative level is determined. Determining whether or not a polypeptide exhibits a particular activity (e.g., determining whether the activity is detectable or not detectable) constitutes determining the activity of the polypeptide in certain embodiments of the invention. In other embodiments a quantitative determination of activity is performed. The phrase “expression or activity” is not intended to indicate that measurements of these parameters are mutually exclusive. A single measurement can provide information about the level of expression, activity, or both. Thus evaluating the level of expression or activity of a protein includes evaluating one or more parameters or features that provide information about the level of expression of the protein, the activity of the protein, or both.

The invention therefore provides methods for evaluating the likelihood that a tumor is sensitive to an mTOR inhibitor comprising assessing the expression or activity of an FKBP protein in a tumor, e.g., in a sample derived from a subject with a tumor or using an in vivo imaging method. In certain embodiments of the invention the level of expression or activity of the FKBP protein is compared with a reference value for the expression or activity of the FKBP protein, and the result of the comparison is predictive of the likelihood that the tumor is sensitive to an mTOR inhibitor.

In accordance with certain embodiments of the invention, a reduced level of expression or activity of an FKBP protein (or its absence) in a tumor, as compared with the reference value indicates a decreased likelihood that the tumor is sensitive to an mTOR inhibitor. In other embodiments, an increased level of expression or activity of the FKBP protein (or its presence) in a tumor, as compared with the reference value indicates an increased likelihood that the tumor is sensitive to an mTOR inhibitor.

The methods may be applied in situations in which a tumor has not been previously exposed to an mTOR inhibitor and in situations in which a tumor has been previously exposed to an mTOR inhibitor and may still be undergoing exposure to the mTOR inhibitor.

A tumor is considered to be sensitive if it is currently displaying sensitivity to an mTOR inhibitor or if it possesses characteristics such that it will display sensitivity to an mTOR inhibitor when exposed to the mTOR inhibitor. A tumor is considered to be resistant if it is currently displaying resistance (lack of sensitivity) to an mTOR inhibitor or if it possesses characteristics such that it will display resistance to the mTOR inhibitor when exposed to the mTOR inhibitor. One of ordinary skill in the art will recognize that a method for evaluating the likelihood that a tumor is sensitive to an mTOR inhibitor also evaluates the likelihood that the tumor is resistant to an mTOR inhibitor. Similarly, one of ordinary skill in the art will recognize that a method for evaluating the likelihood that a subject will exhibit a favorable response to an mTOR inhibitor also evaluates the likelihood that the subject will not exhibit a favorable response to an mTOR inhibitor. For purposes of convenience, the present application refers primarily to methods for evaluating the likelihood that a tumor is sensitive to an mTOR inhibitor and/or that a subject will exhibit a favorable response to an mTOR inhibitor. Such methods are considered equivalent to methods for evaluating the likelihood that a tumor is resistant to an mTOR inhibitor and/or that a subject will not exhibit a favorable response to an mTOR inhibitor since the information obtained by practicing the methods can be expressed in any of these various terminologies.

In certain embodiments of the methods described herein, a sample is obtained from a subject suffering from a tumor. A wide variety of sample types can be used. Typically the sample comprises tumor cells obtained directly from the tumor, e.g., by fine needle aspiration biopsy, tissue biopsy, etc. The sample may be a fresh frozen section, a paraffin embedded section (e.g., a formalin fixed, paraffin embedded section), etc. A tissue sample can be physically disrupted, enzymatically or chemically digested, etc., to obtain individual cells free from surrounding tissue components.

The sample may comprise tumor cells isolated from the blood. Methods for identifying and isolating circulating tumor cells are known in the art (see, e.g., Guadagni, F., et al, Cancer Res. 61:2523-32, 2001; Taback, B., et al., Cancer Res., 61:8845-50, 2001). Such methods may, for example, involve detection of mRNAs (e.g., using RT-PCR) or proteins that are expressed selectively by tumor cells. Many such methods employ antibodies that specifically bind to a protein expressed by the tumor cells, e.g., a cell surface protein, which protein is not expressed by normal cells, particularly cells circulating in the blood (e.g., leukocytes or erythrocytes) or is expressed at a lower level by them. Such proteins are referred to as tumor specific antigens and include, for example, prostate specific antigen (PSA), CA-125, etc. Expression of cytokeratins can also be used to identify tumor cells from epithelial tumors. The level of expression or activity of the FKBP protein in the sample is assessed, and the likelihood that the tumor is sensitive to an mTOR inhibitor is evaluated as described herein. Isolated cells obtained either from a tissue sample, blood sample, aspirate, washing, etc., can be fixed, if desired, and dispersed on a slide or other surface for examination, subjected to flow cytometry, etc.

A reference value for an indicator such as the level of expression or activity of an FKBP protein may be obtained in a variety of ways. In certain embodiments of the invention the reference value is obtained by assessing the indicator in a reference sample or samples. Reference values obtained from any of a number of different reference samples can be used as a basis for comparison with values obtained from a test sample. The nature of the comparison and the interpretation of the result will differ depending on the nature of the reference sample. In some embodiments of the invention cells of the same cell type as the cell type of origin of the tumor, or cells of the same cell type as cells present in the tissue in which the tumor is found or in which the tumor originated, are used as a reference sample. For example, if the tumor is a breast cancer, cells obtained from normal breast tissue can be used as the reference sample. If the tumor is a metastatic (secondary) tumor, cells from the tissue of origin of the primary tumor can be used as a reference sample. For example, if the tumor is a metastasis derived from a primary breast cancer, cells obtained from normal breast tissue may be used as the reference sample. A reference sample comprising normal cells may be obtained from an individual. Typically the individual does not suffer from a tumor. The sample may be obtained from an individual who is undergoing surgery for a condition other than a tumor. Alternately, normal (non-tumor) cells from a subject with a tumor can be used.

In embodiments of the invention in which normal cells are used as the reference sample, if the expression or activity of the FKBP protein in the test sample is lower than the reference value, the tumor is less likely to be sensitive to an mTOR inhibitor (and thus more likely to be resistant to the mTOR inhibitor) than if the expression or activity of the FKBP protein in the test sample is equivalent to or greater than the reference value. If, on the other hand, the expression or activity of the FKBP protein in the test sample is equivalent to or greater than the reference value, the tumor is more likely to be sensitive to the mTOR inhibitor than if the expression or activity of the FKBP protein in the test sample is less than the reference value.

In other embodiments of the invention, cells obtained from a cell line that is sensitive to an mTOR inhibitor in vitro are used as a reference sample. Preferably the cell line is a tumor cell line, although any immortalized cell line can be used. In embodiments in which such cells are used as a reference sample, if the expression or activity of the FKBP protein in the test sample is lower than the reference value, the tumor is less likely to be sensitive to an mTOR inhibitor (and thus more likely to be resistant to the mTOR inhibitor) than if the expression or activity of the FKBP protein in the test sample is equivalent to or greater than the reference value. If, on the other hand, the expression or activity of the FKBP protein in the test sample is equivalent to or greater than the reference value, the tumor is more likely to be sensitive to the mTOR inhibitor than if the expression or activity of the FKBP protein in the test sample is less than the reference value.

In other embodiments of the invention, cells obtained from a cell line that is resistant to an mTOR inhibitor in vitro, are used as a reference sample. Preferably the cell line is a tumor cell line. In embodiments in which such cells are used as a reference sample, if the expression or activity of the FKBP protein in the test sample is lower than or equivalent to the reference value, the tumor is less likely to be sensitive to an mTOR inhibitor (and thus more likely to be resistant to the mTOR inhibitor) than if the expression or activity of the FKBP protein in the test sample is greater than the reference value. If, on the other hand, the expression or activity of the FKBP protein in the test sample is greater than the reference value, the tumor is more likely to be sensitive to the mTOR inhibitor than if the expression or activity of the FKBP protein in the test sample is less than or equivalent to the reference value.

In other embodiments of the invention, tumor cells obtained from a subject who exhibited a favorable response to an mTOR inhibitor following administration of the mTOR inhibitor are used as a reference sample. In embodiments in which such cells are used as a reference sample, if the expression or activity of the FKBP protein in the test sample is lower than the reference value, the tumor is less likely to be sensitive to an mTOR inhibitor (and thus more likely to be resistant to the mTOR inhibitor) than if the expression or activity of the FKBP protein in the test sample is equivalent to or greater than the reference value. If, on the other hand, the expression or activity of the FKBP protein in the reference sample is equivalent to or greater than the reference value, the tumor is more likely to be sensitive to the mTOR inhibitor than if the expression or activity of the FKBP protein in the test sample is less than the reference value.

In other embodiments of the invention, tumor cells obtained from a subject who did not exhibit a favorable response to an mTOR inhibitor following administration of the mTOR inhibitor are used as a reference sample. In embodiments in which such cells are used as a reference sample, if the expression or activity of the FKBP protein in the test sample is lower than or equivalent to the reference value, the tumor is less likely to be sensitive to an mTOR inhibitor (and thus more likely to be resistant to the mTOR inhibitor) than if the expression or activity of the FKBP protein in the test sample is greater than the reference value. If, on the other hand, the expression or activity of the FKBP protein in the test sample is greater than the reference value, the tumor is more likely to be sensitive to the mTOR inhibitor than if the expression or activity of the FKBP protein in the test sample is less than or equivalent to the reference value.

Methods for assessing the level of expression or activity of an FKBP protein in a sample are discussed further below. In other embodiments of the invention the reference value is obtained by an in vivo imaging method that is applied, e.g., to normal tissue, to tumors in subjects that exhibited a favorable response to an mTOR inhibitor, or to tumors in subjects that did not exhibit a favorable response to an mTOR inhibitor. A variety of different imaging modalities may be used to assess expression or activity of an FKBP protein in vivo. For example, a labeled FKBP ligand (e.g., labeled FK506, labeled rapamycin, or a labeled rapamycin analog) may be administered to a subject with a tumor or to a normal individual and its presence in a tumor or in normal tissue may be detected using, e.g., computed tomography (CT) scans, positron emission tomography (PET) scans, etc. Reference values obtained in vivo from normal tissue, from tumors in subjects that exhibited a favorable response to an mTOR inhibitor, or from samples obtained from tumors in subjects that did not exhibit a favorable response to an mTOR inhibitor are used similarly to reference values obtained from samples obtained from normal tissue, from samples obtained from tumors in subjects that exhibited a favorable response to an mTOR inhibitor, or from tumors in subjects that did not exhibit a favorable response to an mTOR inhibitor, respectively.

It will be appreciated that for any of the indicators discussed herein including those related to FKBP proteins and the other indicators discussed below, the determination of a reference value may be performed initially and the value used thereafter for practicing the methods. Thus it is not necessary to determine a reference value or assess a reference sample each time a particular method is practiced. However, in certain embodiments of the invention the test sample is compared with one or more reference or control samples known to display particular expression levels, staining patterns, etc., which are characteristic of sensitive or resistant tumors. Methods for determining a reference value are discussed further below (see Section VII).

In certain embodiments the reference value is a range of values. For example, a tumor may be deemed sensitive to an mTOR inhibitor if a value obtained for an indicator, e.g., the level of expression or activity of the FKBP protein, is within a range of values or is outside a range of values. Conversely, a tumor may be deemed resistant to an mTOR inhibitor if a value obtained for an indicator, e.g., the level of expression or activity of the FKBP protein, is within a range of values or is outside a range of values.

Terms such as “compare”, “comparison” and the like are used broadly herein and include determining whether a value is greater than, equal to, or less than a reference value, determining whether a value falls within a range, etc. An assessment can include determining the extent to which a value differs from a reference value or falls outside a range. An assessment can include determining whether a difference between a value and a reference value is statistically significant.

In certain embodiments of the invention if a test value for an indicator differs from a reference value by a predetermined amount or proportion, the difference is considered informative in term of evaluating the likelihood that a tumor is sensitive to an mTOR inhibitor and/or that a subject will exhibit a favorable response to an mTOR inhibitor. For example, in various embodiments of the invention if a test value is equal to approximately 1%, 5%, 10%, 20%, 30%, 40%, 50%, 60%, 70%, 80%, 90%, 100%, 110%, 120%, 130%, 140%, 150%, 160%, 170%, 180%, 190%, 200%, 250%, 300%, 400%, 500%, 1000%, etc., of the reference value the difference is considered informative, i.e., it indicates an increased or decreased likelihood that the tumor is sensitive and/or that the subject will respond. In other embodiments any difference between a test value and a reference value is considered informative. In certain embodiments a test value that is approximately 5, 10, 15, 20, 25, 50, or more times as large as a reference value is considered informative. Test values that fall within any subrange or have any specific value (specified to the tenths place) within the limits of the values set forth above are considered informative according to various embodiments of the invention. In some embodiments the predetermined amount is independent of the reference and/or test value(s).

Some assessment methods such as immunohistochemistry (discussed further below) utilize a scoring system, e.g., samples are assigned a score ranging from 0-3, from 0-12, etc. When such a scoring system is used, a difference of 1 scoring unit is informative. In other embodiments a difference of 2, 3, 4, 5, or 6 scoring units is considered informative, up to the maximum difference possible according to the scoring system.

It will be appreciated that if the methods are practiced using different techniques for assessing the indicators and/or different instruments, protocols, reagents, etc., the specific reference values used and the extent to which a difference between a test value and a reference value is informative, can differ.

In some embodiments of the invention a relationship is determined between values of an indicator for a tumor or subject and the likelihood that a tumor is sensitive or the subject will exhibit a favorable response. Results of an evaluation can be expressed in terms of the probability (ranging from 0% to 100%) that a tumor having a particular value for an indicator or indicator(s) (e.g., a value for an indicator or indicator(s) that fall(s) within a particular range) is sensitive to an mTOR inhibitor or in terms of the probability that a subject will exhibit a favorable response to an mTOR inhibitor.

In certain embodiments of the invention different reference values are established for each mTOR inhibitor of interest. Alternately, reference values established for one or more mTOR inhibitors may be employed for other mTOR inhibitors, e.g., other mTOR inhibitors that have a similar structure, mechanism of action, etc. For example, a reference value or values established for one rapamycin analog may be used to evaluate the likelihood that a tumor is sensitive to a second rapamycin analog.

If an assessment of the level of an indicator, e.g., the level of expression or activity of an FKBP protein, results in a value indicative of an increased likelihood that a tumor is sensitive to an mTOR inhibitor, the value is referred to as a “favorable value”. If an assessment of an indicator, e.g., the level of expression or activity of an FKBP protein, results in a value indicative of a decreased likelihood that a tumor is sensitive to an mTOR inhibitor, the value is referred to as an “unfavorable value”. In general, the fact that a value is not favorable does not necessarily mean that it is unfavorable, and the fact that a value is not unfavorable does not necessarily mean that it is favorable. In some embodiments of the invention some values are considered uninformative.

The invention further provides methods to evaluate the likelihood that a subject with a tumor will exhibit a favorable response to administration of an mTOR inhibitor. The methods comprise applying any of the methods for evaluating the likelihood that a tumor is sensitive to an mTOR inhibitor to the subject or to a sample derived from the tumor. The likelihood that the tumor is sensitive to an mTOR inhibitor is indicative of the likelihood that the subject will exhibit a favorable response to the mTOR inhibitor or will continue to exhibit a favorable response if he or she is already exhibiting a favorable response.

The methods for evaluating the likelihood that a tumor is sensitive to an mTOR inhibitor and/or for evaluating the likelihood that a subject suffering from a tumor will exhibit a favorable response to an mTOR inhibitor or will continue to respond to an mTOR inhibitor may be performed prior to administering an mTOR inhibitor to a subject or after administration of one or more doses or courses of an mTOR inhibitor to a subject. The invention therefore provides methods for selecting a subject who is a suitable candidate for initiating or continuing treatment with an mTOR inhibitor either as sole therapy or in combination with one or more other agents or treatment modalities such as radiotherapy (gamma-radiation, neutron beam radiotherapy, electron beam radiotherapy, proton therapy, brachytherapy, systemic radioactive isotopes, etc.), surgery, etc., or for selecting an appropriate therapeutic agent or combination of agents and/or other therapeutic modalities for a subject with a tumor.

It is anticipated that mTOR inhibitors may be administered concurrently with one or more additional chemotherapeutic agents either together in single formulation or as individual agents. A “chemotherapeutic agent” is an agent recognized by those of skill in the art as being useful for treating a tumor and/or under investigation or contemplated for such use. The methods of the invention may be used to assess the likelihood that a tumor is sensitive to an mTOR inhibitor and/or the likelihood that a subject will exhibit a favorable response to an mTOR inhibitor when the mTOR inhibitor is administered by itself or concurrently with another chemotherapeutic agent and/or an agent such as an anti-emetic that is administered to counteract side effects of chemotherapy. Such agents include, but are not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), tubulin inhibitors (Vinblastine, Vincristine, Vinorelbine, Paclitaxel (taxol), Docetaxel, epothilones, discodermolides), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), other endocrine therapies, Gleevec™, adriamycin, dexamethasone, cyclophosphamide, biologic response modifiers (e.g., interferons, interleukins, and tumor necrosis factor (TNF)), monoclonal antibodies such as Avastin® (bevacizumab), Herceptin® (trastuzumab), Rituxan® (rituximab), Erbitux® (cetuximab). In certain embodiments the methods are used to predict tumor sensitivity and/or patient response to an mTOR inhibitor administered concurrently with an angiogenesis inhibitor or a kinase inhibitor. In certain embodiments the angiogenesis inhibitor or kinase inhibitor is an anti-VEGF or anti-VEGF-R antibody, small molecule VEGF-R antagonist, anti-angiogenic peptide such as endostatin, an EGF-R antagonist (e.g., SU5416 or SU6668 or an antibody that binds to an EGF-R family member). For a more comprehensive and updated discussion of cancer therapies see, the web site having URL www.nci.nih.gov/, a list of the FDA approved oncology drugs at the web site having URL www.fda.gov/cder/cancer/druglistframe.htm, The Merck Manual, Seventeenth Ed., 1999, and DeVita, supra. It will be appreciated that different reference values may be used in practicing the methods depending the particular therapeutic regimen of the subject.

According to certain of the methods a sample is obtained from a subject who is suffering from a tumor. The likelihood that the tumor is sensitive to the mTOR inhibitor is evaluated and is used as a basis on which to determine whether the subject is a suitable candidate for initiating or continuing treatment with the mTOR inhibitor or for enrolling in or remaining in a clinical trial of the mTOR inhibitor. For example, if the likelihood is greater than a predetermined value, then the subject may be considered a suitable candidate for initiating or continuing treatment with the mTOR inhibitor. If the likelihood is less than a predetermined value, then the subject may be considered not suitable as a candidate. One of ordinary skill in the art will recognize that a variety of factors may be considered in determining whether a subject is a suitable candidate for therapy with an mTOR inhibitor. For example, the subject's response to other therapies, or the results of tests to evaluate the likelihood that the subject will respond to other therapies may be considered as may the side effect profile of the mTOR inhibitor or of any available alternative therapy.

Any of a variety of other indicators may be assessed and used in conjunction with the assessment of the expression or activity of an FKBP protein in order to evaluate the likelihood that a subject having a tumor that is deemed either sensitive or resistant to the mTOR inhibitor will in fact exhibit a favorable response. For example, any of the additional biomarkers described in further detail below may be used. In addition, a variety of other parameters and clinical factors may be considered including, but not limited to, the metabolic activity of the tumor (which may be measured, for example, using functional imaging methods known in the art and discussed below), the size of the tumor, the vascularization of the tumor, whether the tumor has metastasized, the overall health of the subject, prior therapies that the subject has received, etc.

The methods may be used to monitor expression or activity of the FKBP protein over time to determine whether it is appropriate to continue therapy with an mTOR inhibitor and/or to determine whether another agent should be added to the therapeutic regimen. For example, if the level of expression or activity of the FKBP protein decreases over time, or if tumor cell clones emerge that exhibit reduced FKBP expression or activity, it may be advantageous to add another agent to the patient's therapeutic regimen.

### II. FKBP Proteins

Expression or activity of a variety of FKBP proteins may be assessed to evaluate the likelihood that a tumor is sensitive to an mTOR inhibitor. In a preferred embodiment of the invention the FKBP protein is FKBP12, also known as FKBP1A. FKBP12 is described, for example, in the following references (Brown, E. J., et al., Nature 369, 756-758, 1994; Hidalgo et al., Oncogene 19: 6680-6686, 2000). In another preferred embodiment the FKBP protein is FKBP12.6 (Deivanayagam et al., Acta. Crystallogr. D. Biol. Crystallog 56: 266-271, 2000). Other FKBP proteins whose expression or activity may be assessed in accordance with the invention include FKBP51, also referred to as FKBP54 (Nair, et al., Mol. Cell. Biol., 17(2): 594-595, 1997; U.S. Pat. No. 6,821,731) and FKBP52 (Yamamoto-Yamaguchi et al., Exp Hematol 29:582-588, 2001; Genbank Accession No. M88279). FKBP proteins of use in the present invention preferably bind to rapamycin or a rapamycin analog and inhibit the mTOR protein kinase when so bound.

Information concerning the nucleotide sequences, cloning, and other aspects of various FKBP species is known in the art, permitting the synthesis or cloning of DNA encoding any FKBP polypeptide of interest, e.g., using well known methods and PCR primers based on published sequences. See e.g. Staendart et al, 1990, Nature 346, 671-674 (human FKBP12); Kay, 1996, Biochem. J. 314, 361-385 (review). An FKBP coding sequence may be inserted into an expression vector using standard methods so that the coding sequence is operatively linked to expression control sequences such as a promoter, and the expression vector introduced into cells. Such methods may be used, e.g., to express an FKBP in vitro, e.g., to obtain an antigen preparation for immunization of an animal to obtain an antibody to the FKBP protein. An expression vector that encodes an FKBP protein can be used to generate a cell line, e.g., a tumor cell line, that expresses FKBP protein at higher levels than the original cell line. For example, an FKBP protein can be expressed in a tumor cell line that exhibits low sensitivity to an mTOR inhibitor, e.g., is resistant to the mTOR inhibitor, and the sensitivity of the tumor cell line thereby increased.

An FKBP gene sequence can be used to design nucleic acids that inhibit expression of the FKBP protein when introduced into or expressed in cells. Examples include nucleic acids that inhibit gene expression by RNA interference such as short interfering RNA (siRNA) and short hairpin RNA (shRNA). Antisense nucleic acids, which frequently act by inhibiting translation of a complementary mRNA or inducing its degradation by RNase H can also be designed. These inhibitory agents are well known in the art. See, e.g., Dyxhoorn, D., et al., Nat Rev Mol Cell Biol. 4(6):457-67, 2003, for a review that provides sufficient information to allow one of ordinary skill in the art to readily design and test siRNAs and/or shRNAs that will effectively inhibit expression of any gene of interest. See also U.S. Pub. No. 20030108923 and 20040259248. Briefly, an siRNA is typically a short RNA duplex, usually with an ˜19 nucleotide duplex region and symmetric 2-3 nucleotide 3′ overhangs. One strand of the duplex (the antisense or “guide” strand) is complementary to a target mRNA transcript. When present within mammalian cells, siRNAs are incorporated into the endogenous RNA-induced silencing complex (RISC). The siRNA duplex is unwound, and the antisense strand guides RISC to the target mRNA, which is then degraded. One of ordinary skill in the art will appreciate that siRNA are typically chemically synthesized using known methods, and numerous variations on the basic siRNA structure are effective including agents that incorporate a variety of nucleotide analogs and modifications. See, e.g., Crooke, S. (ed.) “Antisense Drug Technology: Principles, Strategies, and Applications” (1^{st} ed), Marcel Dekker; ISBN: 0824705661; 2001) and references therein for discussion of antisense nucleic acids and their mechanism of action. An inhibitory nucleic acid, e.g., an siRNA or siRNA that inhibits expression of an FKBP protein can be introduced into or expressed in cells, e.g., cells of a tumor cell line that is sensitive to an mTOR inhibitor, and the sensitivity of the tumor cell line is thereby decreased.

Tumor cells and tumor cell lines that contain a range of different FKBP expression levels can be generated. Such cells and cell lines can be used, e.g., in screens to identify new chemotherapeutic agents or to explore the effect of varying FKBP expression level on the activity of known chemotherapeutic agents including, but not limited to, mTOR inhibitors such as rapamycin analogs. Such screens may identify therapeutic agents that act synergistically with mTOR inhibitors, that help overcome or prevent resistance to mTOR inhibitors, or that are effective in situations in which mTOR inhibitors are ineffective. The present invention encompasses tumor cells and tumor cell lines with an artificially altered (increased or decreased) expression level of an FKBP protein and methods of using such cells to evaluate candidate therapeutic agents, e.g., by contacting the cells with such agent(s) in the presence or absence of an mTOR inhibitor and evaluating the effect of the agent or combination thereof on cell proliferation. In certain embodiments the tumor cells are implanted into an animal, e.g., a nude mouse, and the effect of one or more candidate anti-tumor agents (e.g., an mTOR inhibitor) is evaluated. Similar methods may be applied for various other indicators described herein. Thus methods of evaluating the likelihood that a tumor is sensitive to an mTOR inhibitor can be applied to tumor cell lines, tumor explants, etc., cultured in vitro, e.g., to aid in the development of new chemotherapeutic agents, in the reanalysis of known agents that did not exhibit sufficient efficacy in clinical trials to merit further development, in the analysis of chemotherapeutic agents currently in use to identify tumor types and phenotypes that are likely to be sensitive, etc.

The various polypeptides of interest herein are referred to by their common names as understood by one of ordinary skill in the art. Sequence information is readily available for each of these proteins, e.g., in public databases such as GenBank. One of ordinary skill in the art will be able to identify the appropriate protein and corresponding nucleic acid sequences for any particular species of interest using the relevant scientific literature and databases. It is noted that frequently a number of entries for each gene, nucleotide, or polypeptide, appear. Such entries are collected under a specific GeneID in GenBank. As known to one of ordinary skill in the art, Gene IDs may be found on the PubMed website (National Library of Medicine). The website may be accessed using URL www.pubmed.com. The GeneID search is performed by selecting “Gene” from the pull-down menu at the top left (below “nucleotide”, “protein”, etc.). The entry for each gene includes considerable information about the gene including links to mRNA and protein sequence entries, links to scientific literature, etc.

The following list presents the names and Gene ID numbers for the human forms of various FKBP proteins. The list also presents names and Gene ID numbers for the human forms of a variety of upstream and downstream components of pathways involving mTOR and also for other polypeptides that are useful as biomarkers for evaluating the likelihood that a tumor is sensitive to an mTOR inhibitor and/or that a subject will exhibit a favorable response to an mTOR inhibitor. If desired, probes and primers (e.g., amplification primers), siRNA sequences, antisense sequences, etc., can readily be designed with no more than routine experimentation based on these sequences. Thus it is possible to amplify, detect, express, inhibit, modify, or otherwise manipulate or utilize nucleic acid sequences that encode any of these proteins and/or the proteins themselves.

FKBP12=2280

FKBP12.6=2281

eIF4E=1977

4E-BP1=1978

4E-BP2=1979

4E-BP3=8637

S6=6194

p70 S6K1=6198

p70 S6K2=6199

mTOR=2475

AKT, 3 isoforms (Akt1-3): 207, 208, 10000

PTEN=5728

TSC2=7249

VHL=7428

Raptor=57521

GβL=64223

IGF-1R=3480

VEGF-A=7422

VEGF-B=7423

VEGF-C=7424

VEGF-D=2277

VEGFR-1=2321

VEGFR-2=3791

HIF-1α=3091

It will be appreciated that in a number of cases a variety of isoforms, family members, splice variants, etc., are referred to collectively using a single name. As used herein, any such name, e.g., AKT, is intended to encompass any individual family member, isoform, or splice variant, and any combination of such family members or isoforms. Thus each embodiment of the instant invention includes variations in which a specific family member, isoform, or splice variant is detected and optionally quantitated to provide information useful for evaluating the likelihood that a tumor is sensitive to an mTOR inhibitor and/or that a subject will exhibit a favorable response to an mTOR inhibitor.

### III. Methods for Assessing Expression or Activity of an FKBP Protein

Any of a variety of methods known to those in the art can be used to assess the level of expression or activity of an FKBP protein in a sample or in a subject. Expression of the protein can be assessed by a direct method, by which is meant any method that is based on detecting and optionally quantitating the protein. In other embodiments expression is assessed by detecting and optionally quantitating mRNA that encodes the protein. In other embodiments expression is assessed by determining whether the genomic DNA of a cell or subject contains a mutation that affects expression of the protein. Typically such a mutation will be in a gene that encodes the protein, e.g., in an expression control region. The mutation may be an insertion, substitution, or deletion of one or more nucleotides. In certain embodiments the mutation is a deletion of part or all of a gene that encodes the FKBP protein.

Methods for detecting and optionally quantitating an FKBP protein typically involve use of a reagent that specifically binds to the FKBP protein. The reagent may be, e.g., an antibody, antibody fragment, aptamer, affibody, polypeptide, small molecule ligand, or the like. Any reagent that specifically binds to the FKBP protein can be used. Such reagents are referred to collectively as “binding agents”. The binding agent may be labeled, e.g., with a radioactive moiety, fluorophore, colorimetric agent, enzyme, magnetically responsive atom or group, etc. In certain embodiments of the invention the FKBP protein is detected and optionally quantitated using an immunological method. Such methods include, but are not limited to, Western blots; immunoassays, e.g., enzyme-linked immunosorbent assays (ELISA); flow cytometry; immunohistochemistry, reverse phase assays, etc. These methods are known to one of ordinary skill in the art and are described, e.g., in U.S. Pat. Nos. 6,821,731; US Pub. Nos. 20030190689, 20040106141, and 20040248151.

Immunohistochemistry (IHC) is a preferred method for detecting and optionally quantitating the level of expression of an FKBP protein and can be performed either manually or using automatic staining instruments. Immunohistochemistry is a method that utilizes monoclonal or polyclonal antibodies to detect cells or specific epitopes. Typically the method detects a protein antigen. Methods for preparing and processing samples for IHC are known in the art. For example, a paraffin-embedded tissue (e.g. tumor tissue) can be prepared for IHC staining by deparaffinizing tissue sections with xylene followed by ethanol; hydrating in water and then PBS; unmasking the antigen by heating the slide in sodium citrate buffer; incubating sections in hydrogen peroxide; blocking in blocking solution; incubating the slide in primary antibody (e.g., antibodies that bind to an FKBP protein, phospho-specific antibodies) and secondary antibody; and finally detecting using an ABC avidin/biotin detection system according to manufacturer's instructions. Numerous variations are possible. Antigen retrieval solutions and methods including microwave treatments, various wash buffers, etc., are known in the art. Methods may be optimized for any particular polypeptide of interest.

Immunohistochemistry protocols employ detection systems that make the epitope detectable to the naked eye, e.g., visible, or detectable to an automated detection system. Typically an antibody (or mixture of antibodies) that binds to a specific protein or other antigen is labeled with a fluorescent or luminescent compound, prosthetic group, radioactive moiety, or an enzyme, e.g., an enzyme that can convert a substrate to a visible dye. The labeled antibody is incubated with the tissue and after washing unbound antibody away the bound antibody distribution is revealed by fluorescence microscopy or incubation with a chromogenic substrate. Alternately the first antibody (primary antibody) can be unlabeled and a second antibody (with an attached label) that binds to the first antibody is employed to render the pattern of antibody staining detectable.

Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material is luminol; examples of bioluminescent materials include luciferase, luciferin, and aequorin. Examples of suitable radioactive material are ¹²⁵I, ¹³¹I, ³⁵S and ³H. Suitable fluorogenic or chromogenic substrates include nitro blue tetrazolium (NBT) in combination with the phosphatase substrate 5-bromo-4-chloro-3-indolyl phosphate (BCIP), diaminobenzidine (DAB), etc.

IHC results can be qualitative, semi-quantitative, or quantitative. In certain embodiments of the invention results are presented in terms of a semi-quantitative scoring system, e.g., ranging from 0 (no staining), to 3+. The score can reflect the percentage of cells that stain, the intensity of staining, the pattern of staining, or any combination of the foregoing. In some embodiments a sample can be considered “positive” or “negative”. For example, a score of 1+, 2+, or 3+ can be considered positive, or 2+ and 3+ can be considered positive with 0 or 1+ being considered negative, etc. Staining in tumor cells can be scored by comparing their intensity of staining with that of endothelial cells present in the sample.

In certain embodiments an automated, quantitative IHC method is used, in which a sample is assigned a score that is a numerical representation of the intensity of the immunohistochemical staining of the sample and represents the amount of the antigen to be detected that is present in the portion of the sample analyzed. The score can be an optical density (OD). Suitable automated IHC sample processing, scanning, and analysis systems are known in the art and are available, e.g., from Ventana Medical Systems. For example, the Benchmark™ system (Ventana Medical Systems, Tuscon Ariz.) performs automated sample preparation and processing. Samples can be assessed visually or optical imaging and computer analysis, e.g., using a system such as the Accumed Accell 2000 Image Analyzer, equipped with appropriate image analysis software, can be used to automatically acquire an image and provide a quantitative measurement of OD.

Flow cytometry is another method that is well known in the art and is useful for assessing expression levels, phosphorylation state, etc., using appropriate antibodies. Flow cytometry may be carried out according to standard methods. See, e.g. Chow et al., Cytometry (Communications in Clinical Cytometry) 46: 72-78 (2001). In certain embodiments of the invention flow cytometry is used to assess the level or proportion of CECs and/or CEPs in a blood sample. Antibodies to cell surface markers that are characteristic of these cell types are known in the art. For example, CD31 and VEGFR-1 identify cells as endothelial cells. CEPs are distinguished by the presence of one or more stem cell antigens on the cell surface such as CD117, Sca-1 or, in humans, CD133. Antibodies to a number of these antigens are commercially available, e.g., from Becton Dickinson. IHC can also be used to assess the level or proportion of CECs and CEPs in a sample.

Laser-scanning cytometry-mediated analysis (LSC) can be used in conjunction with immunofluorescence on tissue biopsy samples to quantify polypeptide expression levels and phosphorylation state on a single cell level (Davis, D W, et al., Clinical Cancer Res., 11:678-689, 2005). Tumor microvessel density (MVD) and vessel sizes can also be measured by LSC (Davis, D W, et al., Cancer Res., 64:4601-10, 2004). Endothelial and/or tumor cell apoptosis can be measured using terminal deoxynucleotidyl transferase-mediated nick end labeling (TUNEL) assays. These assays may provide additional information about the sensitivity of a tumor to an mTOR inhibitor and/or subject response thereto, particularly when administered in conjunction with an angiogenesis inhibitor.

Immunoassays such as ELISA assays or modifications thereof are well known in the art. Information regarding such techniques may be found, for example, in Ausubel, supra, and in Harlow and Lane, supra. Such detection techniques can be divided into (1) antibody capture assays; (2) antigen capture assays; (3) two-antibody capture assays, any of which can be configured by one of ordinary skill in the art to accomplish detection in a qualitative, semi-quantitative, or quantitative mode. One of ordinary skill in the art will be able to select an appropriate assay taking into consideration factors such as the abundance of the molecule to be detected and the relative sensitivity of different assay formats.

In certain embodiments of the invention the level of expression or activity of a protein, e.g., an FKBP protein, is assessed by measuring the expression level of an mRNA that encodes the protein, by determining the presence or absence of a mutation in, or amplification of, a gene that encodes the protein; by determining the presence or absence of a mutation that affects the expression level of the protein and is located outside the gene that encodes the protein. More generally, other methods for assessing the expression or activity of a protein can include determining the existence, number, location, and/or nature of a post-translational modification such as phosphorylation, glycosylation, acetylation, etc.; determining the localization of the protein; or detecting or measuring any biological or chemical activity of the protein (e.g., binding activity, enzymatic activity towards a substrate) etc. A mutation that affects the expression or activity of a protein can be a substitution, deletion, or addition of one or more nucleotides, a chromosomal abnormality such as an inversion, translocation, deletion, rearrangement, amplification, etc.

Suitable methods that may be used to detect and optionally quantitate mRNA include Northern blots, RT-PCR, cDNA or oligonucleotide microarray analysis, in situ hybridization (e.g., fluorescent in situ hybridization), etc. A number of the methods for nucleic acid detection and/or analysis make use of nucleic acid hybridization to detect a nucleic acid of interest, e.g., mRNA, cDNA, or genomic DNA. Suitable probes can readily be designed based on known sequences.

If desired, the nucleic acid of interest can be amplified using methods known in the art. Any suitable amplification method can be used, including exponential amplification, linked linear amplification, ligation-based amplification, and transcription-based amplification. An example of an exponential nucleic acid amplification method is the polymerase chain reaction (PCR) which is described, for example, in Mullis et al. Cold Spring Harbor Symp. Quant. Biol. 51:263-273 (1986); PCR Cloning Protocols: From Molecular Cloning to Genetic Engineering, Methods in Molecular Biology, White, B. A., ed., vol. 67 (1998); Mullis EP 201,184; Mullis et al., U.S. Pat. Nos. 4,582,788 and 4,683,195; Erlich et al., EP 50,424, EP 84,796, EP 258,017, EP 237,362; and Saiki R. et al., U.S. Pat. No. 4,683,194. Linked linear amplification is disclosed by Wallace et al. in U.S. Pat. No. 6,027,923. Examples of ligation-based amplification are the ligation amplification reaction (LAR), taught by Wu et al. (Genomics 4:560 (1989)) and the ligase chain reaction (EP Application No. 0320308 B1). Hampson et al. (Nucl. Acids Res. 24(23):4832-4835, 1996) describe a directional random oligonucleotide primed (DROP) method.

Isothermal target amplification methods include transcription mediated amplification (TMA), self-sustained sequence replication (3SR), Nucleic Acid Sequence Based Amplification (NASBA), and variations thereof. (See Guatelli et al. Proc. Natl. Acad. Sci. U.S.A. 87:1874-1878 (1990); U.S. Pat. Nos. 5,766,849 (TMA); and 5,654,142 (NASBA)) and others (e.g., as described in Malek et al., U.S. Pat. No. 5,130,238; Kacian and Fultz, U.S. Pat. No. 5,399,491; Burg et al., U.S. Pat. No. 5,437,990).

A wide variety of methods are available to detect mutations, e.g., in an FKBP protein or mTOR pathway component. Southern blots and restriction fragment analysis, etc., represent traditional methods. Mutation detection and/or sequence comparison can be performed using any of a variety of methods known in the art, e.g., amplification-based assays, hybridization assays, primer extension assays (e.g., allele-specific primer extension assays), oligonucleotide ligation assays (U.S. Pat. Nos. 5,185,243, 5,679,524 and 5,573,907), cleavage assays, heteroduplex tracking analysis (HTA) assays, etc. Examples include the Taqman® assay, Applied Biosystems (U.S. Pat. No. 5,723,591). In this assay, two PCR primers flank a central probe oligonucleotide. The probe oligonucleotide contains two fluorescent moieties. During the polymerization step of the PCR process, the polymerase cleaves the probe oligonucleotide. The cleavage causes the two fluorescent moieties to become physically separated, which causes a change in the wavelength of the fluorescent emission. As more PCR product is created, the intensity of the novel wavelength increases. Cycling probe technology (CPT), which is a nucleic acid detection system based on signal or probe amplification rather than target amplification (U.S. Pat. Nos. 5,011,769, 5,403,711, 5,660,988, and 4,876,187), could also be employed. Invasive cleavage assays, e.g., Invader® assays (Third Wave Technologies), described in Eis, P. S. et al., Nat. Biotechnol. 19:673, 2001, can also be used to detect mutations and allelic variants. Assays based on molecular beacons (U.S. Pat. Nos. 6,277,607; 6,150,097; 6,037,130) or fluorescence energy transfer (FRET) may be used. Molecular beacons are oligonucleotide hairpins which undergo a conformational change upon binding to a perfectly matched template. The conformational change of the oligonucleotide increases the physical distance between a fluorophore moiety and a quencher moiety present on the oligonucleotide. This increase in physical distance causes the effect of the quencher to be diminished, thus increasing the signal derived from the fluorophore. U.S. Pub. No. 20050069908 and references therein describe a variety of other methods that can be used for the detection and analysis of nucleic acids. U.S. Pat. Nos. 5,854,033, 6,143,495, and 6,239,150 describe compositions and a method for amplification of and multiplex detection of molecules of interest involving rolling circle replication. The method is useful for simultaneously detecting multiple specific nucleic acids in a sample. Of course direct sequencing using any available method known in the art can be used. Examples include the chain termination or dideoxynucleotide method (Sanger, et al., Proc. Natl. Acad. Sci. 74:5463-5467, 1977) and the chemical degradation method (Maxam & Gilbert, Proc. Natl. Acad. Sci. 74:560-564, 1977), of which the former has been most extensively employed, improved upon, and automated. Other sequencing approaches include pyrosequencing (see, e.g., U.S. Pat. Nos. 6,210,891 and 6,258,568; sequencing by hybridization (U.S. Pat. No. 5,202,231; WO 99/60170; WO 00/56937; Drmanac, et al., Advances in Biochemical Engineering/Biotechnology, 77:76-101, 2002), sequencing by synthesis (Melamede, U.S. Pat. No. 4,863,849; Cheeseman, U.S. Pat. No. 5,302,509, Tsien et al, International application WO 91/06678; Rosenthal et al, International application WO 93/21340; Canard et al, Gene, 148: 1-6 (1994); Metzker et al, Nucleic Acids Research, 22: 4259-4267 (1994)), and sequencing by oligonucleotide ligation and detection (U.S. Pat. Nos. 5,740,341 and 6,306,597).

A variety of methods may be used to assess activity of an FKBP protein. Any biological or biochemical activity of the protein can be assessed. In a preferred embodiment the activity is binding activity of the FKBP protein towards mTOR in the presence of an FKBP ligand, e.g., rapamycin or a rapamycin analog. In a preferred embodiment of the invention a labeled small molecule FKBP ligand is employed, e.g. fluorescently labeled form of FK506 such as AP1491. Suitable methods for assaying the binding activity of an FKBP protein, e.g., in a cell lysate, are described in U.S. Pat. Nos. 6,150,527 and 6,649,595.

Similar methods may be employed to assay binding activity of an FKBP protein in a sample such as a tissue biopsy specimen, or in vivo using a suitable imaging technique, e.g., an imaging technique that detects labeled molecules. According to one method a sample, e.g., a tissue biopsy sample, is incubated in the presence of a labeled FKBP ligand. Suitable incubation conditions include, e.g., those described in U.S. Pat. Nos. 6,150,527 and 6,649,595. After a period of time the sample is washed to remove unbound label and is visualized, e.g., using a fluorescence microscope, automated image acquisition system, etc. Similar scoring metrics and/or quantitative analysis can be performed as described above for IHC.

### IV. Additional Biomarkers and Tumor Assessment Methods

A. Biomarkers Based on mTOR Pathway Components

As mentioned above, mTOR is a protein kinase that is a component of a number of signal transduction pathways that regulate a range of cellular processes. In addition to biomarkers based on FKBP proteins, the invention provides a variety of additional indicators that may be assessed either independently or in conjunction with an assessment of the expression or activity of an FKBP protein to evaluate the likelihood that a tumor is sensitive to an mTOR inhibitor. The indicators can be used to evaluate the likelihood that a subject with a tumor will exhibit a favorable response to an mTOR inhibitor. According to certain of the inventive methods the phosphorylation state and/or level of one or more mTOR pathway components is measured, e.g., in a sample derived from a tumor, or using an in vivo imaging method.

As in the case of assessing FKBP protein expression level or activity, a value is obtained for an indicator. The value may be compared with a reference value. General considerations regarding reference values are described in Sections I and VII. In the case of certain indicators the reference value is obtained from a reference sample containing cellular material from any of the sources discussed above, e.g., normal cells (which may be of the same cell type as the cell type of origin of the tumor or of the same cell type as cells presenting the tissue in which the tumor is found), tumor cell lines that are sensitive to an mTOR inhibitor, tumor cell lines that are resistant to an mTOR inhibitor, tumors from subjects who exhibited a favorable response to an mTOR inhibitor, tumors from subjects who did not exhibit a favorable response to an mTOR inhibitor, etc. For other indicators (e.g., level of circulating VEGF, level of CECs, level of CEPs), the reference value is obtained from blood samples from individuals not suffering from a tumor, subjects that exhibited a favorable response to an mTOR inhibitor, or subjects who did not exhibit a favorable response to an mTOR inhibitor. For indicators that are assessed using an imaging study, the reference value can be obtained from normal tissue, from tumors in subjects who exhibited a favorable response to an mTOR inhibitor, or from tumors in subjects who did not exhibit a favorable response to an mTOR inhibitor. If an assessment of an indicator results in a value indicative of an increased likelihood that a tumor is sensitive to an mTOR inhibitor, the value is referred to as a “favorable value”.

Any of a variety of indicators may be assessed and used in conjunction with an assessment of the level of expression or activity of an FKBP protein in order to evaluate the likelihood that a tumor is sensitive to an mTOR inhibitor. The assessment of the level of expression or activity of the FKBP protein and the assessment(s) of one or more additional indicators are considered in conjunction to arrive at an overall assessment of the likelihood that a tumor is sensitive to an mTOR inhibitor. “In conjunction” encompasses a variety of different situations. In various embodiments of the invention the results of each assessment are considered individually, but each of the assessments is used to arrive at an overall assessment. For example, in various embodiments of the invention, if both assessments result in favorable values, the likelihood that the tumor is sensitive is greater than if either or both of the assessments results in an unfavorable value. In other embodiments of the invention results of each assessment are combined (e.g., numerically) to produce a combined value, which is then used to arrive at an overall assessment, e.g., by comparison with a reference value. In certain embodiments none of two or more assessments considered individually is predictive of the likelihood that a tumor is sensitive, but taken collectively the assessments are predictive.

Suitable indicators for use in conduction with the level of expression or activity of an FKBP protein are described below. Certain of these indicators relate to polypeptides that are upstream components of a pathway involving mTOR. As used herein, upstream components of a pathway involving mTOR include IGF-1R, PTEN, AKT, TSC2, and RHEB. Other indicators relate to polypeptides that are downstream components of a pathway involving mTOR. As used herein, downstream components of a pathway involving mTOR include S6, S6 kinase, 4E-BP1, eIF4E, cyclin D1, cyclin D3, myc, p27Kip1, and HIF-1α. Other biomarkers relate to polypeptides that form a complex with mTOR. These polypeptides include Raptor and GβL.

According to the invention the proportion or level of certain upstream or downstream components, or of mTOR itself, that is phosphorylated, is used to evaluate the likelihood that a tumor is sensitive to an mTOR inhibitor. For any given protein, the proportion of that protein that is phosphorylated in a sample can be determined by dividing the amount of that protein that is phosphorylated in the sample by the total amount of that protein present in the sample and multiplying by 100, i.e., by computing 100-[Prot^{P}/(Prot^{P}+Prot^{UP})], where Prot^{P} indicates a phosphorylated form of the protein and Prot^{UP} indicates an unphosphorylated form of the protein. For any given protein, the level of that protein that is phosphorylated in a sample refers to the total amount or concentration of the phosphorylated form of the protein present in a sample. In general, phosphorylation can occur at certain serine, threonine, or tyrosine residues in a protein. Phosphorylation at specific positions is of particular interest in certain embodiments of the invention. For example, phosphorylation at serine 473 of AKT, serine 235 of S6, or serine 2481 of mTOR may be of particular importance. Forms of these proteins that are specifically phosphorylated on these positions are detected in various embodiments of the invention. The proteins may or may not be phosphorylated at one or more other positions in different embodiments.

In certain embodiments of the invention at least one indicator is assessed in a tumor in addition to assessing the level of expression or activity of an FKBP protein. The at least one additional indicator is selected from the group consisting of: (a) the proportion or level of TSC2 protein that is phosphorylated in a tumor; (b) the proportion or level of AKT protein that is phosphorylated in a tumor; (c) the proportion or level of S6 protein that is phosphorylated in a tumor; (d) the proportion or level of S6 kinase protein that is phosphorylated in a tumor; (e) the proportion or level of 4E-BP1 protein that is phosphorylated; (f) the proportion or level of mTOR protein that is phosphorylated; (g) the level of cyclin D1 mRNA or protein, cyclin D3 mRNA or protein, myc mRNA or protein, or any combination of these; (h) the level of HIF-1α mRNA or protein, HIF-1β mRNA or protein, HIF-2α mRNA or protein or any combination of the foregoing; (i) the level of VHL mRNA or protein or presence of a mutation affecting VHL expression or activity; (j) the level of RHEB mRNA or protein or the activity of RHEB protein; (k) the level of eIF4E mRNA or protein; (l) the level of p27Kip1 mRNA or protein; (m) the level of VEGF mRNA or protein; (n) the level of IGF-1R mRNA or protein; (o) the level of PTEN mRNA, protein, or activity, or presence of a mutation affecting PTEN expression or activity; (p) the level of Raptor mRNA, protein, or activity, or presence of a mutation affecting Raptor expression or activity; (q) the level of GβL mRNA, protein, or activity, or presence of a mutation affecting GβL expression or activity; (r) the level of a circulating VEGF polypeptide; and (s) the proportion or level of circulating endothelial cells (CECs), the proportion or level of circulating endothelial progenitor cells (CEPs), or any combination of the foregoing. Indicators (a)-(q) would typically be assessed in a sample containing tumor cells or cellular material. Indicators (r) and (s) refer to cells that are circulating in the bloodstream and would typically be assessed in a blood sample from a subject with a tumor.

Certain of the above indicators relate to assessing the proportion or level of a phosphorylated form of a polypeptide. In certain embodiments of the invention the total level of these polypeptides including both phosphorylated and unphosphorylated forms is assessed instead of, or in addition to, assessing the proportion or level of a phosphorylated form.

According to the invention if one or more of a variety of indicators has a higher value in the tumor than in normal cells or than in tumors that are resistant to an mTOR inhibitor, this difference, in conjunction with a favorable value for the level of expression or activity of an FKBP protein, is indicative of an increased likelihood that the tumor is sensitive to an mTOR inhibitor, relative to the likelihood if the value for that indicator in the tumor is equal to or lower than that in normal cells or in tumors that are resistant to an mTOR inhibitor. The indicators include (a) the proportion or level of TSC2 protein that is phosphorylated; (b) the proportion or level of AKT protein that is phosphorylated; (c) the proportion or level of S6 protein that is phosphorylated; (d) the proportion or level of S6 kinase protein that is phosphorylated; (e) the proportion or level of 4E-BP1 protein that is phosphorylated; (f) the proportion or level of mTOR protein that is phosphorylated; (g) the level of cyclin D1 mRNA or protein, cyclin D3 mRNA or protein, myc mRNA or protein, or any combination of these; (h) the level of HIF-1α mRNA or protein, HIF-1β mRNA or protein, HIF-2α mRNA or protein or any combination of the foregoing; (i) the level of VHL mRNA or protein or a mutation affecting VHL expression; (j) the level of RHEB mRNA or protein or the activity of RHEB protein; (k) the level of eIF4E mRNA or protein; (l) the level of p27Kip1 mRNA or protein; (m) the level of VEGF mRNA or protein; (n) the level of IGF-1R mRNA or protein; (p) the level of Raptor mRNA, protein, or activity; (q) the level of GβL mRNA, protein, or activity; (r) the level of a VEGF mRNA or polypeptide. If the level of PTEN expression or activity in the tumor is lower than in normal cells or in tumors that are sensitive to an mTOR inhibitor, this difference is indicative of an increased likelihood that the tumor is sensitive to the mTOR inhibitor relative to a situation in which the level of PTEN expression or activity in the tumor is equivalent to that in normal cells or in tumors that are resistant to an mTOR inhibitor.

Conversely, if a value for any of indicators (a)-(n) or (p)-(r) is lower in a tumor than in normal cells or tumors that are sensitive to an mTOR inhibitor, this difference, in conjunction with an unfavorable value for the level of expression or activity of an FKBP protein, indicates a decreased likelihood that the tumor is sensitive to the mTOR inhibitor. If the level of PTEN expression or activity in the tumor is equivalent to that in normal cells or in tumors that are resistant to an mTOR inhibitor, this difference, in conjunction with an unfavorable value for the expression or activity of an FKBP protein, is indicative of a decreased likelihood that the tumor is sensitive to the mTOR inhibitor.

In various embodiments of the invention any of the above-mentioned indicators, or any group of two or more of the indicators, is assessed and used in conjunction with an assessment of the level of expression or activity of an FKBP protein to evaluate the likelihood that a tumor is sensitive to an mTOR inhibitor. One or more indicators may be assessed in a single sample, or different samples may be used to assess different indicators.

In other embodiments one or more of the above-mentioned indicators is assessed and used to evaluate the likelihood that a tumor is sensitive to an mTOR inhibitor and/or that a subject will exhibit a favorable response to an mTOR inhibitor. In particular, the invention provides a method for evaluating the likelihood that a tumor is sensitive to an mTOR inhibitor comprising assessing the level of myc mRNA or protein, or any combination of these is assessed. In another embodiment the level of HIF-1α mRNA or protein, HIF-1β mRNA or protein, HIF-2α mRNA or protein or any combination of the foregoing is assessed. In another embodiment the level of VHL mRNA or protein or presence of a mutation affecting VHL expression is assessed. In another embodiment the level of RHEB mRNA or protein or the activity of RHEB protein is assessed. In yet another embodiment the level of Raptor mRNA, protein, or activity, or presence of a mutation affecting Raptor expression or activity, is assessed. In a further embodiment the level of GβL mRNA, protein, or activity, or presence of a mutation affecting GβL expression or activity, is assessed. These indicators may be utilized individually or in conjunction with one or more other indicators to evaluate the likelihood that a tumor is sensitive to an mTOR inhibitor or that a subject will exhibit a favorable response to an mTOR inhibitor.

In certain embodiments of the invention an indicator involving an upstream component of an mTOR pathway is assessed, and the assessment is used in conjunction with an assessment of the level of expression or activity of an FKBP protein to predict the likelihood that a tumor is sensitive to an mTOR inhibitor. In other embodiments of the invention an indicator involving a downstream component of an mTOR pathway is assessed, and the assessment is used in conjunction with an assessment of the level of expression or activity of an FKBP protein to predict the likelihood that a tumor is sensitive to an mTOR inhibitor. In yet other embodiments at least two indicators are assessed in addition to assessing the level of expression or activity of an FKBP protein. For example, a first indicator involving an upstream component and a second indicator involving a downstream component may be used in conjunction with the level of expression or activity of an FKBP protein to predict the likelihood that a tumor is sensitive to an mTOR inhibitor.

Certain of the above-mentioned biomarkers relate to angiogenesis, which is believed to play an important role in maintenance and growth of various tumor types. Polypeptides that are related to angiogenesis include HIF-1α, HIF-1β, HIF-2α, and VEGF. A number of different species and spliced variants of VEGF exist and can be measured either individually or as a group. The term “VEGF” is used herein to refer to any VEGF polypeptide, or any combination of VEGF polypeptides (e.g., VEGF-A, VEGF-B, VEGF-C, VEGF-D, or any combination of the foregoing). In other words, in various embodiments of the invention any VEGF polypeptide or combination thereof may be assessed.

Angiogenesis-related biomarkers include indicators related to the level of expression of HIF-1α, HIF-1β, HIF-2α, or VEGF in a tumor, the level of a circulating VEGF polypeptide in a subject, the proportion or level of circulating endothelial cells (CECs) in a subject, and the proportion or level of circulating endothelial progenitor cells (CEPs) in a subject. The present invention encompasses the recognition that assessments of angiogenesis-related indicators may be of particular value in the context of predicting likelihood of patient response to an mTOR inhibitor. In certain embodiments of the invention an angiogenesis-related indicator and an assessment of the level of expression or activity of an FKBP protein are used in conjunction to evaluate the likelihood that a tumor is sensitive to an mTOR inhibitor and/or the likelihood that a subject will respond to an mTOR inhibitor.

In various embodiments of the invention any group of two or more of the indicators, is assessed and the assessments are used in conjunction with one another to evaluate the likelihood that a tumor is sensitive to an mTOR inhibitor. Optionally an assessment of the expression or activity of an FKBP protein is used in conjunction with assessments of two or more indicators. Particular groups that can be utilized include (a) any indicator related to an upstream component of an mTOR pathway and any indicator related to a downstream component of an mTOR pathway; (b) any indicator related to an upstream component of an mTOR pathway and any angiogenesis-related indicator; (c) any indicator related to a downstream component of an mTOR pathway and any angiogenesis-related indicator; (d) the level of expression or activity of mTOR and any other indicator.

Any of the above methods may be used to evaluate the likelihood that a subject having a tumor will exhibit a favorable response to an mTOR inhibitor.

It is of particular interest to assess the level of expression or activity of an FKBP protein, e.g., FKBP12, in conjunction with assessing one or more of: (a) the proportion or level of TS C2 protein that is phosphorylated; (b) the proportion or level of AKT protein that is phosphorylated; (c) the proportion or level of S6 protein that is phosphorylated; (d) the proportion or level of S6 kinase protein that is phosphorylated; (e) the proportion or level of 4E-BP1 protein that is phosphorylated; and (f) the proportion or level of mTOR protein that is phosphorylated.

It is also of particular interest to assess the level of expression or activity of an FKBP protein, e.g., FKBP12, in conjunction with assessing one or more of: (g) the level of cyclin D1 mRNA or protein, cyclin D3 mRNA or protein, myc mRNA or protein, or any combination of these; (h) the level of HIF-1α mRNA or protein, HIF-1β mRNA or protein, HIF-2α mRNA or protein or any combination of the foregoing.

It is further of particular interest to assess the level of expression or activity of an FKBP protein, e.g., FKBP12, in conjunction with assessing one or more of: (f) the proportion or level of mTOR protein that is phosphorylated; (p) the level of Raptor mRNA, protein, or activity; (q) the level of GβL mRNA, protein, or activity.

It is also of particular interest to assess the level of expression or activity of an FKBP protein, e.g., FKBP12, in conjunction with assessing one or more of: (r) the level of a circulating VEGF polypeptide; and (s) the proportion or level of circulating endothelial cells (CECs), the proportion or level of circulating endothelial progenitor cells (CEPs), or any combination of the foregoing.

The invention further provides methods for predicting or monitoring a subject's response to therapy with an mTOR inhibitor. In certain embodiments one or more of the indicators listed below is/are assessed prior to initiation of treatment with an mTOR inhibitor (e.g., typically within a relatively short time window such as days to 1-2 weeks prior to initiation of therapy) and then again one or times at a relatively early time point in the treatment, e.g., within several hours, days, or weeks after administration of the initial dose of the agent. Typically the indicator is measured after a period of time sufficient to allow the mTOR inhibitor to reach a steady-state plasma concentration, or shortly thereafter. In certain embodiments of the invention the values of an indicator obtained prior to and following administration of an mTOR inhibitor to a subject are compared. The comparison is used to evaluate the likelihood that the tumor is sensitive to an mTOR inhibitor and/or the likelihood that the subject will experience a favorable response to the mTOR inhibitor. In these embodiments the result of the comparison, e.g., the extent to which the value of the indicator changes following administration of the mTOR inhibitor, is compared with a reference value. In preferred embodiments of the invention the reference value in this case is the change in the value of the indicator that occurs following administration of an mTOR inhibitor to subjects who did not exhibit a favorable response to an mTOR inhibitor, or the change in the indicator that occurs following administration of an mTOR inhibitor to subjects who did exhibit a favorable response to the mTOR inhibitor. Certain changes in the value of an indicator following administration of an mTOR inhibitor are indicative of an increased likelihood that the tumor is sensitive to the mTOR inhibitor and/or that the subject is exhibiting or will exhibit a favorable response to the mTOR inhibitor. Such changes are referred to as “favorable alterations”. For example, if a tumor is not sensitive to an mTOR inhibitor, then an indicator of the rate of cell proliferation in the tumor would not be expected to decrease following administration of the mTOR inhibitor, i.e., there would be no alteration, or there would be an increase in the value of the indicator. If the tumor is sensitive to the mTOR inhibitor, then an indicator of the rate of cell proliferation in the tumor would be expected to decrease following administration of the mTOR inhibitor. Such an alteration would be a favorable alteration in the indicator.

A favorable alteration in one or more indicators is indicative of an increased likelihood that the tumor is sensitive to the mTOR inhibitor and, in general, is indicative of an increased likelihood that the subject is exhibiting or will exhibit a favorable response to the mTOR inhibitor, relative to the likelihood of such response if the favorable alteration is not observed.

In certain embodiments one or more of the following indicators is/are evaluated in a tumor or in a subject with a tumor: (a) the proportion or level of TSC2 protein that is phosphorylated; (b) the proportion or level of AKT protein that is phosphorylated; (c) the proportion or level of S6 protein that is phosphorylated; (d) the proportion or level of S6 kinase protein that is phosphorylated; (e) the proportion or level of 4E-BP1 protein that is phosphorylated; (f) the proportion or level of mTOR protein that is phosphorylated; (g) the level of cyclin D1 mRNA or protein, cyclin D3 mRNA or protein, myc mRNA or protein, or any combination of these; (h) the level of HIF-1α mRNA or protein, HIF-2α mRNA or protein; HIF-1β mRNA or protein, or any combination of the foregoing (i) the level of VHL mRNA or protein or a mutation affecting VHL expression; (j) the level of RHEB mRNA or protein or the activity of RHEB protein; (k) the level of eIF4E mRNA or protein; (l) the level of p27Kip1 mRNA or protein; (m) the level of VEGF-R mRNA or protein; (n) the level of IGF-1R mRNA or protein; (o) the level of PTEN mRNA, protein, or activity, or a mutation affecting PTEN expression; (p) the level of Raptor mRNA, protein, or activity, or a mutation affecting Raptor expression; (q) the level of GβL mRNA, protein, or activity, or a mutation affecting GβL expression; (r) the level of a VEGF mRNA or polypeptide; (s) the level of a circulating VEGF polypeptide; and (t) the level of circulating endothelial cells (CECs), the level of circulating endothelial progenitor cells (CEPs), or both.

The following alterations following administration of an mTOR inhibitor are indicative of an increased likelihood that a tumor is sensitive to the mTOR inhibitor and are considered favorable alterations: (a) a decrease in the proportion or level of TSC2 protein that is phosphorylated; (b) a decrease in the proportion or level of AKT protein that is phosphorylated; (c) a decrease in the proportion or level of S6 protein that is phosphorylated; (d) a decrease in the proportion or level of S6 kinase protein that is phosphorylated; (e) a decrease in the proportion or level of 4E-BP1 protein that is phosphorylated; (f) a decrease in the proportion or level of mTOR protein that is phosphorylated; (g) a decrease in the level of cyclin D1 mRNA or protein, cyclin D3 mRNA or protein, myc mRNA or protein, or any combination of these; (h) a decrease in the level of HIF-1α mRNA or protein, HIF-2α mRNA or protein; HIF-1β mRNA or protein, or any combination of the foregoing; (i) a decrease in the level of VEGF mRNA or protein (e.g., VEGF-A, VEGF-B, VEGF-C, VEGF-D); (j) a decrease in the level of a circulating VEGF polypeptide (e.g., VEGF-A, VEGF-B, VEGF-C, VEGF-D); and (k) a decrease in the level or proportion of circulating endothelial progenitor cells (CEPs). In certain embodiments of the invention an indicator based on detecting an upstream component of an mTOR pathway (e.g., TSC2, AKT, mTOR) and an indicator based on detecting a downstream component of an mTOR pathway (e.g., S6, S6 kinase, cyclin D1, cyclin D3, myc, 4E-BP1) are assessed. Any combination of indicators can be assessed. In certain embodiments of the invention the level of expression or activity of an FKBP protein is assessed and its value is used in conjunction with a favorable or unfavorable alteration in one or more of the above indicators to evaluate the likelihood that a tumor is sensitive to an mTOR inhibitor.

The size of a favorable alteration can vary. For example, a favorable alteration can be an increase or decrease of at least approximately 5%, 10%, 20%, 30%, 40%, 50%, 60%, 60%, 80%, 90%, 100%, 150%, 200% (2-fold), 300% (3-fold), 4-fold, 5-fold, 10-fold, etc., of the pre-administration value for the indicator. Any subrange or specific value (specified to the tenths place) within these ranges is a favorable alteration according to various embodiments of the invention. A favorable alteration can be a change from a value that indicates cell proliferation to a value that indicates a lower degree of cell proliferation, i.e., a lower cell proliferation rate. For example, a decline in standard uptake value in a PET scan is a favorable alteration in certain embodiments of the invention.

It is also of particular interest to assess the level of expression or activity of an FKBP protein, e.g., FKBP12, in conjunction with assessing a change that occurs in one or more of the following after administration of an mTOR inhibitor: (a) the proportion or level of TSC2 protein that is phosphorylated; (b) the proportion or level of AKT protein that is phosphorylated; (c) the proportion or level of S6 protein that is phosphorylated; (d) the proportion or level of S6 kinase protein that is phosphorylated; (e) the proportion or level of 4E-BP1 protein that is phosphorylated; (f) the proportion or level of mTOR protein that is phosphorylated.

It is also of particular interest to assess the level of expression or activity of an FKBP protein, e.g., FKBP12, in conjunction with assessing a change that occurs in one or more of the following after administration of an mTOR inhibitor: (g) the level of cyclin D1 mRNA or protein, cyclin D3 mRNA or protein, myc mRNA or protein, or any combination of these; (h) the level of HIF-1α mRNA or protein, HIF-1β mRNA or protein, HIF-2α mRNA or protein or any combination of the foregoing.

It is also of particular interest to assess the level of expression or activity of an FKBP protein, e.g., FKBP12, in conjunction with assessing a change that occurs in one or more of the following after administration of an mTOR inhibitor: (f) the proportion or level of mTOR protein that is phosphorylated; (j) the level of RHEB mRNA or protein or the activity of RHEB protein; (o) the level of PTEN mRNA, protein, or activity, or a mutation affecting PTEN expression; (p) the level of Raptor mRNA, protein, or activity, or a mutation affecting Raptor expression; (q) the level of GβL mRNA, protein, or activity, or a mutation affecting GβL expression. Of course a change in any of the above indicators may be assessed in conjunction with assessing the level of expression or activity of an FKBP protein, e.g., FKBP12.

Methods for assessing the proportion or level of a phosphorylated protein include immunological methods that make use of phospho-specific antibodies, i.e., antibodies that detect a phosphorylated form of a protein but generally do not substantially detect unphosphorylated forms. Suitable immunological methods include Western blots, immunoassays, and IHC, which are described above. A phospho-specific antibody may specifically detect a form of the protein that is phosphorylated at a particular position. Suitable phospho-specific antibodies are known in the art and are described, e.g., in U.S. Pub. Nos. 20030190689, 20040106141, and 20040248151. These antibodies can be used in assays such as Western blots, immunoassays, immunohistochemistry, etc., according to conventional methods of performing these assays. Additional phospho-specific antibodies can be obtained by immunizing an animal with a phosphorylated protein or portion of a protein that contains a phospho-epitope, and identifying or purifying proteins that bind specifically to the phosphorylated form of the protein or portion thereof and do not bind to the unphosphorylated form and/or to forms that are phosphorylated at a position other than the position of interest. Table 1 presents a list of antibodies that may be used in the practice the inventive methods, and suppliers thereof.

TABLE 1

| Protein | Vendor | Catalog # |
|---|---|---|
| PTEN | Zymed | 18-0256 |
| Akt | Novus | 8805 |
| Phospho-Akt (Ser 473) | Cell Signaling | 3787 |
| | Technology | |
| S6 | Cell Signaling | 2217 |
| | Technology | |
| Phospho-S6 (Ser235/236) | Cell Signaling | 2211 |
| | Technology | |
| 4E-BP1 | Novus | NB200-157 |
| Phospho-4E-BP1 | Cell Signaling | 9455 |
| (Ser65/Thr70) | Technology | |
| Cyclin D1 | VMSI | 760-2935 |
| p27 | Neomarkers | RB-9019-P |
| IGF-1R | Neomarkers | MS641-P1 |
| VEGF | Santa Cruz | 7269 |
| | Biotechnology | |
| elF4E | Cell Signaling | 9742 |
| | Technology | |
| mTOR | Abcam | 2732 |
| Phospho-mTOR (Ser2448) | Cell Signaling | 2971 |
| | Technology | |
| TSC2 | Upstate | 07-456 |
| Phospho-TSC2 (Tyr1571) | Cell Signaling | 3614 |
| | Technology | |
| FKBP12 | Alexis | ALX-210-142 |
| S6 kinase | Cell Signaling | 9202 |
| | Technology | |
| Phospho-S6 kinase | Cell Signaling | 9205 |
| (Thr389) | Technology | |
| Cyclin D3 | Cell Signaling | 2936 |
| | Technology | |
| myc | Santa Cruz | 789 |
| | Biotechnology | |
| HIF-1α | Cell Signaling | 3716 |
| | Technology | |
| HIF-1β | Abcam | 2771 |
| HIF-2α | Abcam | 13704 |
| VHL | Santa Cruz | 5575 |
| | Biotechnology | |
| RHEB | Santa Cruz | 6342 |
| | Biotechnology | |
| Raptor | Cell Signaling | 4978 |
| | Technology | |
| GBL | GenWay Biotech | A21973 |

Various of the indicators involve detecting or measuring an activity of one or more components of an mTOR pathway. Methods of assessing an activity are selected in accordance with the particular activity. For example, AKT, IGF-1R, S6 kinase, and mTOR are kinases, and their kinase activity can be measured by performing a kinase assay using an appropriate substrate or by examining the amount of a phosphorylated substrate in a sample using a phosphor-specific antibody. Relevant natural and synthetic substrates and methods for performing kinase assays are known in the art. RHEB is a GTPase, and an appropriate method for assessing its activity is a GTPase assay. PTEN is a phosphatase, and an appropriate method for assessing its activity is a phosphatase assay. See, e.g., Rahman, A., et al., Bioassay Techniques for Drug Development, Harwood Academic Publishers, Amsterdam, 2001, for description of a large number of cell-based assays.

Raptor and GβL form complexes with mTOR, and an appropriate assay for their activity is a binding assay, e.g., a co-immunoprecipitation assay, immunoaffinity assay, competition assay, etc. Activity can also be assessed by determining whether a mutation exists in an active site or in a region of the protein needed for activity. For example, phosphorylation sites are known to be important for activity of certain of the kinases mentioned above. Mutations at these sites will generally diminish activity of the kinase. Mutations that affect the activity of other molecules mentioned above are known in the art or can be determined by sequencing DNA from tumors in which such activity is altered. An exemplary mutation is a mutation in a region of mTOR that interacts with the FKBP12/rapamycin complex, e.g., the FRB region (Chen, et al., Proc. Natl. Acad. Sci. USA, 92: 4947-4951, 1995; Choi, et al., Science, 273:239-42, 1996), which may alter the binding of an FKBP/inhibitor complex to mTOR.

B. Angiogenesis-Related Biomarkers

The invention provides a variety of biomarkers that reflect the degree of vascularization of a tumor or the degree of angiogenic activity of the tumor. Certain of these biomarkers are of use for evaluating the likelihood that a tumor is sensitive to an mTOR inhibitor. Certain additional angiogenesis-related biomarkers are of use for monitoring the response of a subject to therapy with an mTOR inhibitor. The invention provides a method of evaluating the likelihood that tumor is sensitive to an mTOR inhibitor comprising measuring the level of CEPs in a subject prior to administration of an mTOR inhibitor, administering the mTOR inhibitor to the subject, and measuring the level of CEPs in the subject within a short time window (e.g., days to weeks) following administration of the mTOR inhibitor, wherein a decrease in the number of CEPs following administration of the mTOR inhibitor is indicative of an increased likelihood that the subject has a tumor that is sensitive to the mTOR inhibitor. Methods for identifying CECs and CEPs based on their expression of particular cell surface proteins are known in the art (Ribatti, D., J. Cell. Mol. Med., Vol. 8, No. 3: 294-300, 2004; Shaked, Y., et al., Cancer Cell, Vol. 7:101-111, 2005). Flow cytometry can be used in a representative embodiment.

The invention also provides a method of evaluating the likelihood that tumor is sensitive to an mTOR inhibitor comprising measuring the level of circulating VEGF polypeptide in a subject prior to administration of an mTOR inhibitor, administering the mTOR inhibitor to the subject, and measuring the level of circulating VEGF polypeptide in the subject within a short time window (e.g., days to weeks) following administration of the mTOR inhibitor, wherein a decrease in the amount of VEGF polypeptide following administration of the mTOR inhibitor is indicative of an increased likelihood that the subject has a tumor that is sensitive to the mTOR inhibitor. In alternative embodiments the level of VEGF in the tumor is measured, wherein a decrease in VEGF in the tumor is indicative of an increased likelihood that the subject has a tumor that is sensitive to the mTOR inhibitor. Methods for evaluating the angiogenesis-related indicators include methods for detecting mRNA or protein discussed above, and functional imaging methods such as those discussed below.

In certain embodiments an angiogenesis-related indicator and a second indicator based on detecting an alteration in the proportion or level of phosphorylation of an mTOR pathway component are measured. A favorable alteration in both indicators indicates an increased likelihood that the tumor is sensitive to the mTOR inhibitor relative to the likelihood in which there is a favorable alteration in only one of the indicators.

It is of particular interest to assess the level of expression or activity of an FKBP protein, e.g., FKBP12, in conjunction with assessing an angiogenesis-related indicator.

C. Functional Imaging

The invention encompasses the use of various imaging techniques to assess the ability of an mTOR inhibitor to inhibit tumor growth in vivo. The methods may be used to identify a subject who is a suitable candidate for therapy with an mTOR inhibitor and/or to evaluate or predict the likelihood that a subject will exhibit a favorable response to an mTOR inhibitor. Conventional imaging modalities, including conventional X-rays, conventional CT scans, or conventional magnetic resonance imaging (MRI) scans, rely on detecting changes in tumor mass and volume that can take weeks or months to become evident. In preferred embodiments of the invention a functional imaging technique is used. A functional imaging technique detects a feature of the tumor that is indicative of its growth rate or state such as cell metabolism, cell proliferation, blood flow, etc. The invention provides the recognition that functional imaging is of particular use in selecting patients for therapy with an mTOR inhibitor, for predicting the likelihood of response, and/of for monitoring therapy. Of course it is to be understood that a functional imaging technique may also detect changes in tumor mass and volume in addition to providing functional information, e.g., information related to the physiological state and/or physiological processes of the tumor cells.

Positron emission tomography using a labeled metabolic substrate or precursor is a preferred functional imaging technique. For example, ¹⁸F 2-fluoro-2-deoxyglucose ([¹⁸F]-FDG) positron emission tomography (FDG-PET) can be used to measure cellular metabolic activity since the rate of [¹⁸F]-FDG accumulation in tumors is proportional to the rate of glucose utilization (Gupta, N., et al., Eur. J. Cancer, 38:2094-2107, 2002). The European Organization for Research and Treatment of Cancer (EORTC) PET study group has developed guidelines for assessing tumor sensitivity and/or subject response (Young, H., et al., Eur. J. Cancer, 35:1773-1782, 1999). In certain embodiments of the invention these guidelines or updated versions thereof are used to assess tumor response to administration of an mTOR inhibitor. PET methodology is also available for measuring radiolabeled thymidine (e.g., 2-[¹¹C] thymidine or 3′-deoxy-3′-[¹⁸F] fluorothymidine (FLT) incorporation into DNA to provide an assessment of cell proliferation and thus tumor growth rate in vivo.

The invention also encompasses use of PET to measure angiogenesis, hypoxia, and/or apoptosis in a tumor prior to and/or following administration of an mTOR inhibitor. VEGF is upregulated in a number of tumor types. Angiogenesis can be measured, e.g., by PET, after administering a labeled antibody to VEGF. Blood flow can be measured by PET after administration of labeled (e.g., ¹⁵O-labeled) H₂O and/or CO. One of ordinary skill in the art will recognize that a variety of functional imaging methods utilizing alternative substrates, precursors, and detection modalities can also be used. For example, functional CT and functional MRI are capable of detecting blood flow and metabolic activity in tumors using appropriate contrast agents, etc. (see, e.g., Padhani, A., Eur. J. Cancer, 38:2116-2127, 2002).

In certain embodiments of the invention an image that provides functional information about a tumor (a “functional image”) is obtained from a subject prior to administration of an mTOR inhibitor. Typically the image is obtained within a relatively short window of time prior to administration of the mTOR inhibitor (e.g., immediately before (within hours), up to several (e.g., 1-4) days before, up to 1 week before, or up to several weeks before administration of an mTOR inhibitor. The mTOR inhibitor is then administered to the subject, and a second functional image is obtained. Preferably the second functional image is obtained a relatively short time after administration of the mTOR inhibitor, e.g., after the mTOR inhibitor has reached a steady-state plasma concentration, after several days, after approximately 1 week, 1-2 weeks, up to 4 weeks, etc., after administration of the mTOR inhibitor. A significant decrease in a parameter indicative of cell metabolism and/or cell proliferation (e.g., a decrease in standard uptake values (SUV) for [¹⁸F]-FDG relative to the SUV prior to administration of the mTOR inhibitor) is indicative of an increased likelihood that the tumor is sensitive to the mTOR inhibitor and is indicative of an increased likelihood that the subject is exhibiting or will exhibit a favorable response to the mTOR inhibitor, relative to the likelihood of sensitivity and/or response if there is no decrease in the parameter. The invention encompasses any method in which a functional image of a tumor is obtained prior to and following administration of an mTOR inhibitor, e.g., a rapamycin analog, to a subject with the tumor.

It is of particular interest to assess the level of expression or activity of an FKBP protein, e.g., FKBP12, and to utilize the assessment in conjunction with an assessment based on performing an imaging study prior to and following administration of an mTOR inhibitor to a subject. For example, it is of particular interest to assess the level of expression or activity of an FKBP protein, e.g., FKBP12, and to assess a change in tumor metabolism and/or cell proliferation, e.g., using PET. In particular, a favorable value for FKBP protein expression or activity in conjunction with a favorable alteration in SUV (or in another measurement of tumor cell metabolism and/or proliferation) may indicate an increased likelihood that a subject will exhibit a favorable response to an mTOR inhibitor, relative to the likelihood in a situation in which the subject does not have a favorable value for the FKBP protein and/or does not exhibit a favorable alteration in SUV (or another measurement of tumor cell metabolism and/or proliferation)

It is further of particular interest to assess the level of expression or activity of an FKBP protein, e.g., FKBP12, and to utilize the assessment in conjunction with both (i) an assessment based on performing an imaging study prior to and following administration of an mTOR inhibitor to a subject and (ii) a second indicator of the likelihood that a tumor is sensitive to an mTOR inhibitor and/or that a subject will exhibit a favorable response to an mTOR inhibitor, wherein the second indicator is any of the non image-based indicators discussed herein.

### V. mTOR Inhibitors

The present invention may be used to evaluate the likelihood that a tumor is sensitive to any of a variety of mTOR inhibitors and/or to evaluate the likelihood that a subject will respond to any of a variety of mTOR inhibitors. In preferred embodiments the mTOR inhibitor is a rapamycin analog, which term includes rapamycin derivatives such as those described below.

Rapamycin is a macrolide antibiotic produced by Streptomyces hygroscopicus and discovered in the 1970's. Rapamycin is a potent immunosuppressive agent and is used clinically to prevent rejection of transplanted organs. It has also been reported to have activity as an antifungal agent, in the experimental allergic encephalomyelitis model (a model for multiple sclerosis), in the adjuvant arthritis model (for rheumatoid arthritis), in inhibiting the formation of IgE-like antibodies, and for treating or preventing lupus erythematosus, pulmonary inflammation, insulin dependent diabetes mellitus, adult T-cell leukemia/lymphoma, smooth muscle cell proliferation and intimal thickening following vascular injury as well as certain cancers. See e.g. US Pat. appln 2001/0010920. In addition to their role in chemotherapy, rapamycin and everolimus are used as immunosuppressants in organ transplant recipients, and rapamycin and a number of the C-43-modified rapamycin analogs are being used, evaluated or developed for use on stents as anti-restenotic agents following interventional cardiology.

The following structural diagram illustrates rapamycin and a few C-43-substituted derivatives of rapamycin, where the “R” group is located at C-43 in our numbering convention.

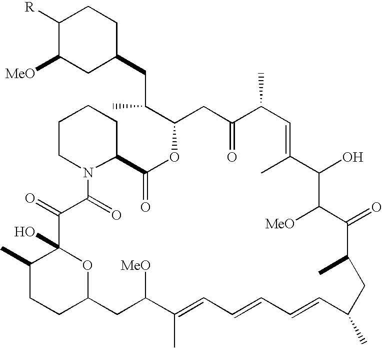

| R | Compound |
|---|---|
| HO— | rapamycin |
| (Me)₂P(O)—O— | AP23573 |
| (HOCH₂)₂C(CH₃)CO₂— | temsirolimus |
| HOCH₂CH₂O— | everolimus |

These compounds are non-limiting examples of potent mTOR inhibitors. For additional information on AP23573, see WO 03/064383. For a recent reference on temserolimus (CCI779), see WO 2004/026280. For everolimus, see U.S. Pat. No. 6,384,046 and references cited therein.

Many other rapamycin derivatives modified at position 43 and/or at one or more other positions are known. For instance, certain other O-substituted rapamycins are disclosed in WO 94/02136, U.S. Pat. No. 5,258,389 and WO 94/09010 (O-aryl and O-alkyl rapamycins); see also WO 92/05179 (carboxylic acid esters), U.S. Pat. No. 5,118,677 (amide esters), U.S. Pat. No. 5,118,678 (carbamates), U.S. Pat. No. 5,100,883 (fluorinated esters), U.S. Pat. No. 5,151,413 (acetals), U.S. Pat. No. 5,120,842 (silyl ethers), WO 93/11130 (methylene rapamycin and derivatives), WO 94/02136 (methoxy derivatives), WO 94/02385 and WO 95/14023 (alkenyl derivatives). Certain dihydro or substituted rapamycin derivatives are described, e.g., in U.S. Pat. No. 5,256,790 See also U.S. Pat. No. 6,710,053. Further rapamycin derivatives are described in PCT application number EP96/02441, for example 32-deoxorapamycin as described in Example 1 therein, and 16-pent-2-ynyloxy-32(S)-dihydrorapamycin as described in Examples 2 and 3 therein (using that document's numbering system).

For additional non-limiting examples analogs and derivatives of rapamycin see, e.g., WO 01/144387 and the references in Table 2.

TABLE 2

| | | | | |
|---|---|---|---|---|
| WO 97/10502 | WO 94/18207 | WO 93/04680 | U.S. Pat. No. 5,527,907 | U.S. Pat. No. 5,225,403 |
| WO 96/41807 | WO 94/10843 | WO 92/14737 | U.S. Pat. No. 5,484,799 | U.S. Pat. No. 5,221,625 |
| WO 96/35423 | WO 94/09010 | WO 92/05179 | U.S. Pat. No. 5,457,194 | U.S. Pat. No. 5,210,030 |
| WO 96/03430 | WO 94/04540 | U.S. Pat. No. 5,604,234 | U.S. Pat. No. 5,457,182 | U.S. Pat. No. 5,208,241 |
| WO 96/00282 | WO 94/02485 | U.S. Pat. No. 5,597,715 | U.S. Pat. No. 5,362,735 | U.S. Pat. No. 5,200,411 |
| WO 95/16691 | WO 94/02137 | U.S. Pat. No. 5,583,139 | U.S. Pat. No. 5,324,644 | U.S. Pat. No. 5,198,421 |
| WO 95/15328 | WO 94/02136 | U.S. Pat. No. 5,563,172 | U.S. Pat. No. 5,318,895 | U.S. Pat. No. 5,147,877 |
| WO 95/07468 | WO 93/25533 | U.S. Pat. No. 5,561,228 | U.S. Pat. No. 5,310,903 | U.S. Pat. No. 5,140,018 |
| WO 95/04738 | WO 93/18043 | U.S. Pat. No. 5,561,137 | U.S. Pat. No. 5,310,901 | U.S. Pat. No. 5,116,756 |
| WO 95/04060 | WO 93/13663 | U.S. Pat. No. 5,541,193 | U.S. Pat. No. 5,258,389 | U.S. Pat. No. 5,109,112 |
| WO 94/25022 | WO 93/11130 | U.S. Pat. No. 5,541,189 | U.S. Pat. No. 5,252,732 | U.S. Pat. No. 5,093,338 |
| WO 94/21644 | WO 93/10122 | U.S. Pat. No. 5,534,632 | U.S. Pat. No. 5,247,076 | U.S. Pat. No. 5,091,389 |

Other mTOR inhibitors include, but are not limited to, small molecule or antibody inhibitors of any of the kinases in the mTOR pathway (e.g., AKT, PI3 kinase), e.g., wortmannin, LY compounds, etc. Other inhibitors disrupt the interaction between GβL and mTOR, or between Raptor and mTOR. Agents such as siRNA or antisense molecules that reduce expression of mTOR, AKT, eIF4E, Raptor, or GβL, are additional examples of mTOR inhibitors.

### VI. Kits, Databases, and Diagnostic Instruments

The invention provides reagents and kits for evaluating the likelihood that a tumor is sensitive to an mTOR inhibitor and/or for evaluating the likelihood that a subject with a tumor will exhibit a favorable response to an mTOR inhibitor. The kits comprise a validated reagent for assessing the expression or activity of an FKBP protein in a sample or in a subject. By “validated” is meant that the reagent has been used to assess the level of expression or activity of an FKBP protein in a plurality of samples derived from tumors that have been exposed to an mTOR inhibitor, and/or in a plurality of subjects with tumors to whom an mTOR inhibitor has been administered. Preferably it has been demonstrated that the results obtained using the reagent are reproducible and consistent. Preferably it has been demonstrated that the results obtained using the reagent are of use for evaluating the likelihood that a tumor is sensitive to the mTOR inhibitor and/or that a subject will experience a favorable response to an mTOR inhibitor.

In certain preferred embodiments of the invention the reagent is an antibody, antibody fragment, antibody derivative, or ligand that specifically binds to the FKBP protein. In other embodiments the reagent is a nucleic acid that hybridizes to a nucleic acid that encodes the FKBP protein. In certain embodiments the FKBP protein is FKBP12. The antibody, antibody fragment, antibody derivative, or ligand may be labeled. For example, labeled FK506, labeled rapamycin, or a labeled rapamycin analog such as AP1491 (fluoresceinated FK506) can be used. In certain embodiments the kit further comprises at least one item selected from the group consisting of: a positive control sample, a negative control sample, one or more reference samples, a substrate, an enzyme, a wash solution, a reagent for assessing a second indicator of the likelihood that a tumor is sensitive to an mTOR inhibitor; and instructions for use of the kit. The reagent for assessing a second indicator of the likelihood that a tumor is sensitive to an mTOR inhibitor may be a validated reagent.

In other embodiments the kit comprises a reagent for assessing the expression or activity of an FKBP protein in a sample or in a subject and further comprises at least one item selected from the group consisting of: a reagent for assessing a second indicator of the likelihood that the tumor is sensitive to an mTOR inhibitor, a positive control sample, a negative control sample, one or more reference samples, a substrate, an enzyme, a wash solution, and instructions for use of the kit. Either or both of the reagents may be a validated reagent.

In certain embodiments of the invention a kit contains one or more antibodies or other binding agents, each of which specifically binds to a polypeptide selected from the group consisting of: IGF-R1, PTEN, AKT, TSC2, S6, S6 kinase, mTOR, GβL, Raptor, 4E-BP1, HIF-1α, or any of the other components of an mTOR pathway discussed herein. In certain embodiments one or more of the antibodies is a phospho-specific antibody. For example, an antibody that binds to a phosphorylated form of AKT, S6, S6 kinase, 4E-BP1, TSC2, mTOR, etc., can be included. In some embodiments an antibody that recognizes phosphorylated or unphosphorylated forms of one or more of the aforementioned polypeptides. Suitable antibodies are available, e.g., from Cell Signaling Technologies (Beverly, Mass.).

In any of the kits the positive control sample may be, e.g., cells that express the FKBP protein. The negative control may be, e.g., cells in which the FKBP protein is not expressed at a level that is detectable using the kit or is expressed at a low level. The reference sample may be, e.g., cells from a tumor that is sensitive to an mTOR inhibitor, cells from a tumor that is resistant to an mTOR inhibitor, etc. The positive, negative, and/or reference sample may comprise cells provided on a slide.

Kits will generally include one or more vessels, tubes, or containers so that certain of the individual kit components may be separately housed. The kits may also include a means for enclosing the individual containers in relatively close confinement for commercial sale, e.g., a plastic or Styrofoam box, in which instructions, packaging materials, etc., may be enclosed. Containers or substrates, e.g., slides, microtiter dishes, etc., for performing an assay may be included. The kits and/or reagent containers may have a bar code or radio frequency (RF) ID tag.

For a variety of purposes, e.g., in order to effectively evaluate and/or treat patients, etc., it will often be desirable to store and/or analyze data such as values for one or more indicators and information regarding samples or subjects using a computer and appropriate software. The invention therefore provides a computer-readable medium (e.g., hard or floppy disk, zip disk, CD, flash memory, or the like) on which is stored: (a) a value for each of one or more indicators selected from the group consisting of: the level of expression or activity of an FKBP protein; the proportion or level of TSC2 protein that is phosphorylated; the proportion or level of AKT protein that is phosphorylated; the proportion or level of S6 protein that is phosphorylated; the proportion or level of S6 kinase protein that is phosphorylated; the proportion or level of 4E-BP1 protein that is phosphorylated; the proportion or level of mTOR protein that is phosphorylated; the level of cyclin D1 mRNA or protein, cyclin D3 mRNA or protein, myc mRNA or protein, or any combination of these; the level of HIF-1α mRNA or protein, HIF-2α mRNA or protein; HIF-1β mRNA or protein, or any combination of the foregoing; the level of VHL mRNA or protein or a mutation affecting VHL expression; the level of RHEB mRNA or protein or the activity of RHEB protein; the level of eIF4E mRNA or protein; the level of p27Kip1 mRNA or protein; the level of VEGF-R mRNA or protein; the level of IGF-1R mRNA or protein; the level of PTEN mRNA, protein, or activity, or a mutation affecting PTEN expression; the level of Raptor mRNA, protein, or activity, or a mutation affecting Raptor expression; the level of GβL mRNA, protein, or activity, or a mutation affecting GβL expression; the level of a circulating VEGF polypeptide; and the level of circulating endothelial cells (CECs), the level of circulating endothelial progenitor cells (CEPs), or both; and a value obtained from a functional imaging study, wherein the value for the one or more indicators was obtained from a sample derived from the tumor or from a subject suffering from the tumor; and (b) tumor-related information indicating (i) whether or not the tumor is sensitive to an mTOR inhibitor, (ii) whether or not a subject having the tumor exhibited a clinical response to an mTOR inhibitor, or (iii) both, wherein the value and the information are associated with one another such that it is possible to determine which indicator was obtained from which sample, tumor, or subject.

The values and information may be stored in any convenient format. In general, the computer-readable medium will store values for one or more indicators, wherein the values are obtained from a plurality of different samples derived from tumors or from subjects having a tumor. The values and information are associated with each other in the sense that the computer-readable medium stores sufficient information so that it is possible to determine for each tumor, sample, or subject for which an indicator value is stored, the tumor-related information relevant to that tumor, sample, or subject. Preferably the values and tumor-related information are stored in a database, e.g., a relational database. For any particular tumor, sample, or subject, it is possible to retrieve the indicator values obtained from that tumor, sample, or subject, and the tumor-related information for that tumor, sample, or subject. Typically the samples, tumors, and/or subjects will be identified by an identifier, e.g., an alphanumeric code, bar code reference number or bar code character string, name, social security number, etc.

A large variety of additional data elements may be stored. For example, patient-related information such as would typically be found in medical records, e.g., age, sex, medical history, medications, etc., can be stored. Information related to specific therapeutic regimens and protocols can also be included.

The invention encompasses a computer or other instrument that comprises a computer-readable medium as described above. The invention further encompasses an automated staining machine, e.g., for performing IHC to assess one or more of the indicators described herein, e.g., to assess the level or activity of an FKBP protein, e.g., FKBP12.

### VII. Statistical Methods for Identifying Indicators and Correlations and Determining Reference Values

The indicators described herein are generally based on the identification of differences between groups, such that a difference in the indicator between different groups correlates with a physiologically relevant characteristic or feature, e.g., a difference in sensitivity to an mTOR inhibitor. For example, groups of tumors that express different levels of an FKBP protein may display differential sensitivity to an mTOR inhibitor, and groups of subjects with tumors that express different levels of an FKBP protein may display differential responsiveness to an mTOR inhibitor such that there is a difference in the response rate between groups of subjects whose tumors express different levels of an FKBP protein. Statistical methods known to one of ordinary skill in the art can be used to demonstrate a statistically significant difference for an indicator between groups and to demonstrate a correlation with a characteristic or feature of interest, e.g., a therapeutically relevant characteristic such as sensitivity to an mTOR inhibitor. Generally a statistically significant difference is one that has a less than 5% likelihood of occurring by chance.

Suitable methods for demonstrating correlations include t-tests (e.g., Fisher t test), chi square tests, determination of the Pearson correlation coefficient, linear regression, cluster analysis, etc. See, e.g., Motulsky, H., Intuitive Biostatistics (ISBN 0-19-508607-4), Oxford University Press Inc., 1995, and US Pub. No. 20030190689. One of ordinary skill in the art will recognize that selection of a suitable statistical test will depend upon the question being asked, type of data, etc., and that suitable tests are selected for any particular experiment or trial. Exemplary tests and appropriate situations for their use are listed in Table 3. Software for performing statistical tests is widely available. Software packages such as SAS, Systat, Sigmastat, Graphpad, R software (Ihaka, R. and Gentleman, R. R., J. Comput. Graphical Statistics, 5:229-314, 1996) etc., can be used. For dividing tumors and/or subjects into different groups based on multiple indicators, cluster analysis can be performed. Programs for performing hierarchical clustering (Cluster and Treeview) are publicly available at the Stanford University web site or at the web site having URL rana.lbl.gov/EisenSoftware.htm and widely known in the art (initially described in Eisen M. B., et al. Proc. Natl. Acad. Sci. USA 95:14863-14868, 1998 and subsequently modified).

In certain embodiments of the invention a relationship between the value(s) of one or more indicator(s) and the likelihood that a tumor is sensitive to an mTOR inhibitor and/or the likelihood that a subject with a tumor will exhibit a favorable response to an mTOR inhibitor. The relationship may be determined using regression in the case of a single indicator or multiple regression in the case of multiple indicators. The relationship can be linear or nonlinear. In one embodiment a multivariate logistic regression model is used (Choe, G., et al., Cancer Res., 63:2742-2746, 2003).

TABLE 2

| | Type of Data | | | |
|---|---|---|---|---|
| | | Rank, Score, or | | |
| | | Measurement | Binomial | |
| | Measurement | (from Non- | (Two | |
| | (from Gaussian | Gaussian | Possible | |
| Goal | Population) | Population) | Outcomes) | Survival Time |
| Describe one | Mean, SD | Median, | Proportion | Kaplan Meier |
| group | | interquartile range | | survival curve |
| Compare one | One-sample t | Wilcoxon test | Chi-square | |
| group to a | test | | or Binomial test** | |
| hypothetical | | | | |
| value | | | | |
| Compare two | Unpaired t test | Mann-Whitney test | Fisher's test | Log-rank test |
| unpaired | | | (chi-square | or Mantel- |
| groups | | | for large | Haenszel* |
| | | | samples) | |
| Compare two | Paired t test | Wilcoxon test | McNemar's | Conditional |
| paired groups | | | test | proportional |
| | | | | hazards |
| | | | | regression* |
| Compare three | One-way | Kruskal-Wallis test | Chi-square | Cox proportional |
| or more | ANOVA | | test | hazard |
| unmatched | | | | regression** |
| groups | | | | |
| Compare three | Repeated- | Friedman test | Cochrane Q** | Conditional |
| or more | measures | | | proportional |
| matched | ANOVA | | | hazards |
| groups | | | | regression** |
| Quantify | Pearson | Spearman | Contingency | |
| association | correlation | correlation | coefficients** | |
| between two | | | | |
| variables | | | | |
| Predict value | Simple linear | Nonparametric | Simple logistic | Cox |
| from another | regression | regression** | regression* | proportional |
| measured | or | | | hazard |
| variable | Nonlinear | | | regression* |
| | regression | | | |
| Predict value | Multiple linear | | Multiple | Cox |
| from several | regression* | | logistic | proportional |
| measured or | or | | regression* | hazard |
| binomial | Multiple | | | regression* |
| variables | nonlinear | | | |
| | regression** | | | |

A variety of statistical methods known to one of ordinary skill in the art may likewise be used to determine reference values for practicing the methods herein. Typically determination of a reference value involves assessing a plurality of samples having certain characteristics of interest in common and combining the values obtained therefrom in any of a number of different ways to arrive at a value that is characteristic of the plurality and that correlates with a characteristic or feature of interest, e.g., a therapeutically relevant characteristic such as sensitivity to an mTOR inhibitor. For example, a mean, median, mode, or other statistic can be computed. Not all of the values need to be considered in arriving at the reference value. For example, outliers may be excluded. One of ordinary skill in the art is aware of appropriate statistical methods for obtaining a reference value from a plurality of values.

In certain embodiments of the invention, a reference value for an indicator is obtained from one or more groups of samples or subjects. For example, depending upon the particular indicator and methods for its assessment, a reference value may be obtained from a plurality of (a) samples containing normal cells; (b) samples from tumors that are sensitive to an mTOR inhibitor; (c) samples from tumors that are resistant to an mTOR inhibitor; (d) samples from tumors in subjects who did exhibited a favorable response to an mTOR inhibitor; (e) samples from tumors in subjects who did not exhibit a favorable response to an mTOR inhibitor, (f) blood samples from subjects not having a tumor; (g) blood samples from subjects who exhibited a favorable response to an mTOR inhibitor; (h) blood samples from subjects who did not exhibit a favorable response to an mTOR inhibitor, etc. In the case of imaging studies, reference values can be obtained from (i) normal tissue; (o) tumors in subjects who exhibited a favorable response to an mTOR inhibitor; (k) tumors in subjects who did not exhibit a favorable response to an mTOR inhibitor. The number of samples or subjects assessed in each group can vary, e.g., from as few as 2 to dozens, hundreds, thousands, etc. While it is generally preferred to obtain reference values from multiple samples or subjects, in some embodiments of the invention a reference value is obtained from a single sample or subject.

In some embodiments of the invention a reference value for an indicator is obtained from a tumor cell line, e.g., any of the tumor cell lines listed in Example 1. For example, a reference value obtained from a tumor cell line that is relatively sensitive to an mTOR inhibitor can be used, or a reference value obtained from a tumor cell line that is relatively resistant to an mTOR inhibitor can be used.

In certain embodiments of the invention an indicator, e.g., an FKBP expression or activity level, is measured in archived tumor samples, particularly from subjects who have been treated with an mTOR inhibitor. The results are correlated with the likelihood that a subject having the tumor exhibited a favorable response to treatment and are used to obtain appropriate reference values for practicing one or more of the methods herein.

Any of the indicators may be assessed in samples provided in array format, e.g., as “tissue microarrays”, optionally including automated systems for processing the arrays. Tissue microarrays typically comprise dozens to hundreds of cylindrical tissue biopsies (diameter 0.6 mm) from morphologically representative regions of individual tumors, which are arrayed in a single paraffin block. Consecutive sections from such arrays provide targets for parallel in situ visualization and/or quantitation of DNA, RNA or protein targets. See, e.g., U.S. Pub. Nos. 20020192702 and 20030215936. The samples may or may not be from subjects who were treated with an mTOR inhibitor. Analysing large numbers of tumor samples, e.g., by assessing the level of FKBP protein or assessing one or more of the other indicators described herein, allows the identification of correlations between values for these indicators and particular histopathologic types, grades, clinical stages, etc., and/or allows the identification of correlations between indicators.

### VIII. Methods of Treatment

This invention thus also provides a method for treating a subject suffering from a tumor, the method comprising the steps of (1) evaluating the likelihood that the tumor is sensitive to an mTOR inhibitor; and (2) administering an mTOR inhibitor to the subject. The invention further provides a method for treating a subject suffering from a tumor, the method comprising the steps of (1) evaluating the likelihood that the subject will exhibit a favorable response to an mTOR inhibitor; and (2) administering an mTOR inhibitor to the subject. In either of the foregoing methods, preferably the step of evaluating is performed according to one of the methods described herein.

The step of evaluating may comprise evaluating one or more of any of the indicators described herein. For example, the step of evaluating may comprise assessing the expression level or activity of an FKBP protein, e.g., FKBP12, in a sample derived from the subject in need of such evaluation, or assessing an indicator of the expression or activity of the FKBP protein in the tumor in vivo, wherein the value of the indicator, relative to a reference value for the indicator, is predictive of the likelihood that the tumor is sensitive to the mTOR inhibitor. In other embodiments, the step of evaluating comprises (a) assessing the expression level or activity of an FKBP protein, e.g., FKBP12, in a sample derived from the subject in need of such evaluation, or assessing an indicator of the expression or activity of the FKBP protein in the tumor in vivo, wherein the value of the indicator, relative to a reference value for the indicator, is predictive of the likelihood that the tumor is sensitive to the mTOR inhibitor; and (b) assessing at least one additional indicator of the likelihood that the tumor is sensitive to an mTOR inhibitor, wherein the value of the at least one additional indicator, relative to a reference value for the indicator, is predictive of the likelihood that the tumor is sensitive to the mTOR inhibitor. The at least one additional indicator may be any of the indicators mentioned herein.

Optimally the practice of the above methods of treatment will constitute treating a subject selected for a higher likelihood that his or her tumor is sensitive to an mTOR inhibitor and/or that the subject will respond to an mTOR inhibitor relative, for example, to the likelihood of such sensitivity and/or response in the overall population of tumor and subjects. Preferably the result of such treatment is beneficial to the overall health of the subject.

Preferred mTOR inhibitors include but are not limited to Serolimus, Temsirolimus, Everolimus, and AP23573. Preferably the mTOR inhibitor of steps (1) and (2) of the above methods is the same, e.g., the likelihood of sensitivity to a specific mTOR inhibitor is evaluated, and that particular mTOR inhibitor is administered to the subject. Of special interest is the application of the foregoing methods to patients with one of the various sarcomas; breast cancer; endometrial cancer; prostate cancer; a leukemia, lymphoma or other hematologic cancer; or a brain cancer, although the invention is applicable to a far wider range of cancers as discussed herein. As mentioned above, it is contemplated that mTOR inhibitors may be administered as part of a therapeutic regimen that includes one or more additional chemotherapeutic agents and/or as part of a therapeutic regimen that includes a treatment modality such as radiation therapy, surgery, etc. The methods of treatment encompass these situations. Thus in certain embodiments the methods may involve administration of one or more additional chemotherapeutic agents.

### EXAMPLES

### Example 1

### Correlation Between FKBP12 Expression Level and Sensitivity to a Rapamycin Analog in Tumor Cell Lines

Materials and Methods

Determination of the Degree of Sensitivity of Cell Lines to AP23573.

A series of in vitro proliferation assays were performed on 14 human tumor-derived cell lines exposed to AP23573 at concentrations ranging from 0.01 nM to 100 mM. The cell lines are listed in Table 4. Cells were plated into 96-well plates (between 750 and 5000 cells per well as per the table below), in cell culture medium containing serial dilutions of AP23573, or in the absence of AP23573 as a control, and were then incubated for 3 days.

At the end of the incubation, the number of cells in each well (cell viability) was determined using a CellTiter 96® Aqueous One Solution Cell Proliferation metabolic activity assay kit (Promega). This assay is based on the use of a reagent that monitors the total reducing environment of a population of cells. The quantity of reduced products, which is determined spectrophotometrically, is directly proportional to the number of viable cells in the culture. The percentage of cells remaining after treatment with each concentration of AP23573 was then determined relative to the number of cells remaining in the untreated wells.

For each cell line the percentage cells remaining after treatment with AP23573 reached a plateau between 1 and 10 nM. Thus the inhibitory effect of AP23573 on cell proliferation was calculated by taking the average of the percentage of cells remaining at the 10, 33 and 100 nM concentration levels. A lower percentage of cells remaining reflects a larger inhibitory effect of AP23573. The experiment was performed two or three times for each cell line and the results averaged.

TABLE 4

| | | | Cell per | Cell per |
|---|---|---|---|---|
| | | ATCC | 96-well | 10 cm dish |
| Cell line | Tissue origin | catalog # | well | (×10E5) |
| HT1080 | Fibrosarcoma | CCL-121 | 1250 | 1.875 |
| AN3 CA | Endometrium | HTB-111 | 2500 | 3.75 |
| PC-3 | Prostate | CRL-1435 | 2500 | 3.75 |
| ES-2 | Ovarian | CRL-1978 | 750 | 1.125 |
| SK-BR-3 | Breast | HTB-30 | 2500 | 3.75 |
| MCF-7 | Breast | HTB-22 | 2500 | 7.5 |
| MDA-MB-453 | Breast | HTB-131 | 5000 | 7.5 |
| SK-OV-3 | Ovarian | HTB-77 | 1250 | 1.875 |
| MDA-MB-435S | Breast | HTB-129 | 2500 | 3.75 |
| HEC-1-A | Endometrium | HTB-112 | 2500 | 3.75 |
| MDA-MB-468 | Erythroleukemia | HTB-132 | 2500 | 3.75 |
| BT-474 | Colon | HTB-20 | 5000 | 7.5 |
| DU 145 | Prostate | HTB-81 | 2500 | 3.75 |
| HCT-116 | Breast | CCL-247 | 2500 | 3.75 |

Determination of the Level of FKBP12 Expression in Cell Lines.

A series of immunoblot studies was performed to determine the levels of FKBP12 protein in all 14 cell lines. Cells were plated in a 10 cm dish at approximately the same cell density used in the study to measure the effects of AP23573 on cell proliferation (the number of cells plated for each cell line is indicated in the table). After 3 days of incubation (in the absence of AP23573), cell lysates were prepared and samples containing 35 μg total protein were separated by sodium dodecyl sulfate polyacrylamide gel electrophoresis. Proteins were transferred to a membrane for immunoblotting, and the presence of FKBP12 was determined by probing with an anti-FKBP12 antibody (Affinity Bioreagents # PA1-026A) followed by incubation with a labelled secondary antibody and autoradiography, using a standard immunoblotting procedure. The membranes were stripped and reprobed with an antibody against the GAPDH protein (Abcam # ab9485) as a control. Quantitation was performed by scanning the X-ray film and analyzing the data using 1D Image Analysis Software (Kodak).

Results

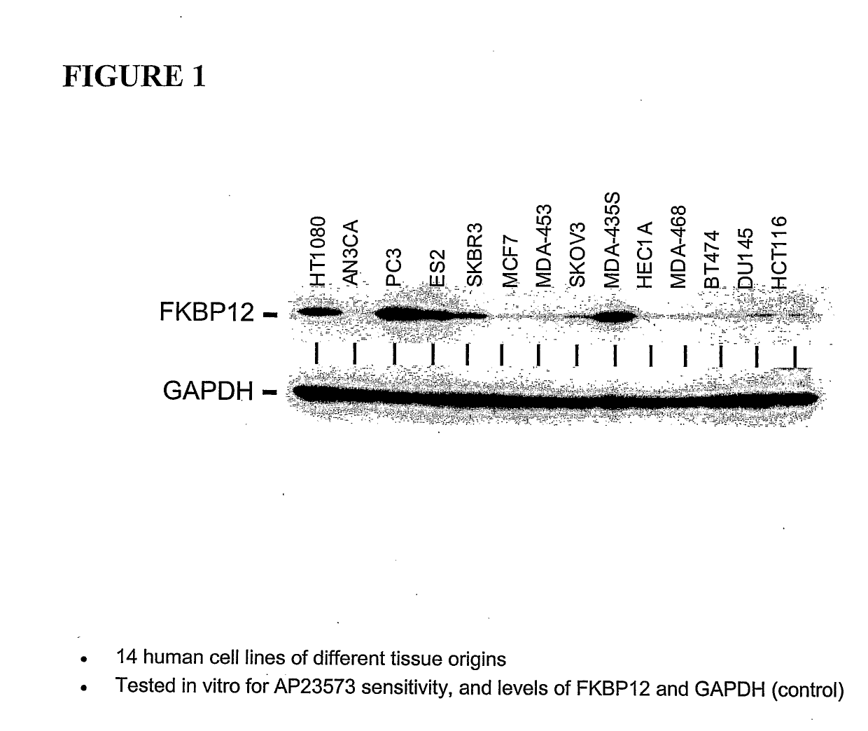

FIG. 1 shows the FKBP12 expression levels in the cell lines. Table 5 shows the correlation between expression levels of FKBP12 and sensitivity to AP23573. In general, cell lines having higher expression of FKBP12 relative to other cell lines were more sensitive to AP23573 than cell lines with lower levels of FKBP12 expression as evidenced by the fact that a greater percentage of cells remained after treatment in the case of cell lines that had lower levels of FKBP12 expression and a lower percentage of cells remained after treatment in the case of cell lines that had higher levels of FKBP12. For example, HT1080, PC3, ES2, SKBR3 display relatively high levels of FKBP12 expression and relatively high levels of sensitivity, whereas HEC1A, BT474, MDA468, DU145, and HCT116 display lower levels of FKBP12 expression and lower levels of sensitivity to AP23573. Cell lines having lower levels of expression of FKBP12 relative to other cell lines were generally less sensitive to AP23573. Thus higher expression of FKBP12 is positively correlated with sensitivity to an AP23573.

TABLE 5

| | FKBP levels | |
|---|---|---|
| | (relative | Percent Cells |
| Cell line | units) | Remaining |
| HT1080 | 313329 | 37 |
| AN3CA | 31683 | 45 |
| PC3 | 495316 | 51 |
| ES2 | 351634 | 63 |
| SKBR3 | 183325 | 55 |
| MCF7 | 19989 | 55 |
| MDA-453 | 20675 | 58 |
| SKOV3 | 77109 | 61 |
| MDA-435S | 411134 | 68 |
| HEC1A | 15901 | 70 |
| MDA-468 | 23343 | 77 |
| BT474 | 9205 | 80 |
| DU145 | 56665 | 81 |
| HCT116 | 48678 | 94 |

### Example 2

### Effect of Expressing FKBP12 in a Tumor Cell Line that is Resistant to a Rapamycin Analog on mTOR Sensitivity

Cell line HCT116 is relatively resistant to AP23573 and displays a low FKBP12 expression level. To confirm that an increase in FKBP12 expression level would confer increased sensitivity to AP23573, HCT116 cells are transfected with an FKBP12 expression vector. The human FKBP12 ORF is obtained by PCR using the cDNA IMAGE clone, CS0DG001YF135, as a template (Invitrogen). The hFKBP ORF is ligated into the SalI and NotI sites of p4694 to generate pFKBP12. The plasmid is purified using the Qiagen (Valencia, Calif.) Megaprep endotoxin-free kit according to the manufacturer's directions and verified by sequencing analysis. Transfection is performed using standard procedures. Populations of pFKBP12-transfected and control cells are divided into aliquots for parallel analysis of FKBP12 expression and AP23573 sensitivity.

Protein extracts from aliquots of pFKBP12-transfected cells and control cells are tested to assess their FKBP12 protein levels by SDS-polyacrylamide gel (10% or 4-20% gradient gel) and Western blot analyses. Mouse antihuman FKBP12 antibody (BD Transduction Laboratories, San Diego) is used at 1:500 dilution. FKBP12 levels are quantified as described in Example 1. Observation of a greater level of FKBP12 protein in pFKBP12-transfected cells relative to controls (cells transfected with p4694 lacking an insert) confirms that the transfectants express high levels of FKBP12.

Real-time quantitative PCR is performed as an alternative method to assess FKBP12 expression level. Total RNA from aliquots of pFKBP12-transfected cells is isolated a using Nucleospin RNAII kit (Clontech). Five μg of total RNA is treated with 20 units of DNase and reverse transcribed with oligo dT:random primer (1:1 at 1.5 μg each) by using Superscript II (Invitrogen). Fifty nanograms of first-strand cDNA is used in subsequent real-time PCR carried out with GeneAmp 5700 (Applied Biosystems) by using SyBr green dye (Applied Biosystems) as the fluorescent probe. The following gene-specific primer sets, generated by using PRIMER EXPRESS (Applied Biosystems) are used: FKBP12 sense primer 5′-AGATGAGTGTGGGTCAGAGA-3′, FKBP12 antisense primer 5′-TAGAAG CTCCACATCGAAGAC-3′. The following parameters are used in the PCR: 10 min denaturation at 95° C., 35 cycles of 15 sec at 95° C., and 1 min at 55° C. To quantitate expression in pFKBP12-transfected cells, the C_{T} values are used. C_{T} is the point at which the fluorescence signal rises above the baseline fluorescence and begins to increase exponentially. The C_{T} value is in logarithmic inverse relationship with the abundance of the transcripts, based on the assumption that C_{T} values increase by ≈1 for each twofold dilution. Detection of an greater level of FKBP12 mRNA in pFKBP12-transfected cells relative to controls (cells transfected with p4694 lacking an insert) confirms that the transfectants express high levels of FKBP12 mRNA and, presumptively, high levels of FKBP12 protein.

As a third means to assess FKBP12 expression, an in vitro binding assay is performed on lysates from aliquots of pFKBP12-transfected cells and control cells using the fluorescein-labeled FKBP12 ligand AP1491. The assay is performed essentially as described in U.S. Pat. Nos. 6,150,527 and 6,649,595.

To examine sensitivity to AP23573, a series of in vitro proliferation assays are performed on populations of control cells and populations of pFBKP12-transfected cells exposed to AP23573 at concentrations ranging from 0.01 nM to 100 nM. The assays are performed as described in Example 1. The percentage of cells remaining is determined. A lower percentage of pFKBP12-transfected cells remaining than control cells remaining after treatment at a particular AP23573 concentration is an indication that higher expression of FKBP12 results in increased sensitivity to AP23573. The correlation between FKBP12 expression levels and sensitivity to AP23573 is evaluated using linear regression.

In addition, at 24, 48, and 72 h posttransfection, pFKBP12-transfected and control cells are harvested for DNA content analysis as described (Lamm, G. M., et al., Nucleic Acids Res. 25, 4855-4857, 1997). Cells are analyzed to determine the proportion of cells arrested in the G1 phase of the cell cycle. Cell-cycle analysis was performed using FACScan (Becton Dickinson) and CELLQUEST and MODFITLT analysis programs (Becton Dickinson). A higher proportion of FKBP12-transfected cells in G1 phase than control cells in G1 phase is an indication that higher expression of FKBP12 results in increased sensitivity to AP23573. The correlation between FKBP12 expression levels and sensitivity to AP23573 is evaluated using a linear regression model. Differences between sensitivity in pFKBP12-transfected and control cells are analysed using at test to determine whether an increase in FKBP12 expression results in an increase in sensitivity. A P value <0.05 is considered statistically significant. Statistical analysis is performed using InStat Statistical Software version 3.05 (San Diego, Calif.).

### Example 3

### Effect of Reducing Expression of FKBP12 Using siRNA Targeted to FKBP12 in a Tumor Cell Line that is Sensitive to a Rapamycin Analog on mTOR Sensitivity

Cell line HT1080 cells are relatively sensitive to AP23573 and displays a high FKBP12 expression level. To confirm that an decrease in FKBP12 expression level would result in decreased sensitivity to AP23573, FKBP12 expression in HT1080 cells is inhibited using RNA interference by transfecting the cells with a FKBP12-specific siRNA. siRNA construction and transfection is performed as follows: FKBP12 siRNA is synthesized in vitro using the Silencer short interfering RNA (siRNA) construction kit (Ambion, Austin, Tex.), according to the manufacturer's instructions. The sense DNA oligo used in in vitro transcription is: 5′-AATAGGCATAGTCTGAGGAGACCTGTCTC-3′. A negative control siRNA sequence is designed by randomizing the above oligonucleotide sequence to obtain 5′-CCTGGGTAGTCTAAATAGAAGGTAGCCTC-3′ and the negative control siRNA is synthesized according to the same protocol as the FKBP12 siRNA. A total of 25 nM siRNA is transfected using the siPORT lipid transfection reagent (Ambion) according to the manufacturer's instructions.

Populations of FKBP12 siRNA-transfected cells and control cells are divided into aliquots for parallel analysis of FKBP12 expression and AP23573 sensitivity. FKBP12 expression is assessed as described in Example 2. Aliquots of cells are treated with AP23573 and their sensitivity is assessed using a cell proliferation assay and cell cycle analysis as described in Example 2. A higher percentage of FKBP12 siRNA-transfected cells remaining than control cells remaining after treatment at a particular AP23573 concentration is an indication that decreasing the expression of FKBP12 results in decreased sensitivity to AP23573. The correlation between FKBP12 expression levels and sensitivity to AP23573 is evaluated using linear regression. A lower proportion of FKBP12 siRNA-transfected cells in G1 phase than control cells in G1 phase is an indication that lower expression of FKBP12 results in decreased sensitivity to AP23573.

The correlation between FKBP12 expression levels and sensitivity to AP23573 is evaluated using a linear regression model. Differences between sensitivity in pFKBP12-transfected and control cells are analysed using at test to determine whether an increase in FKBP12 expression results in an increase in sensitivity. A P value <0.05 is considered statistically significant. Statistical analysis is performed using InStat Statistical Software version 3.05 (San Diego, Calif.).

### Example 4

### Correlation Between FKBP12 Expression Level and Patient Response to a Rapamycin Analog

To confirm the clinical significance of FKBP12 expression levels in evaluating the likelihood of favorable response to treatment with an mTOR inhibitor, FKBP12 expression is evaluated in 50 tumor samples (tissue biopsy samples) from subjects who responded favorably to an mTOR inhibitor and 50 tumor samples from subjects who did not exhibit a favorable response after similar treatment. The experiment is repeated several times using either mixed sets of samples that included samples from tumors of various types or using sets of samples from a single tumor type (prostate cancer, leiomyosarcoma, osteosarcoma). Some of the samples are obtained from an ongoing clinical trial of AP23573 in sarcoma, results of which are presented below:

| | | | | | | Overall |
|---|---|---|---|---|---|---|
| | | | | | | Response (CR + |
| Disease Cohort (N) | | CR/PR | SD | PD | NE/TE | PR + SD) |
| Bone Cohort | (19) | 3 | 11 | 5 | 2 | 14/19 (74%) |
| Leiomyosarcoma | (18) | 0 | 12 | 6 | 2 | 12/18 (60%) |
| Liposarcoma | (13) | 0 | 8 | 5 | 8 | 8/13 (61%) |
| Other Sarcoma | (20) | 0 | 13 | 7 | 0 | 13/20 (65%) |

N = those patients with at least one response assessment
CR = complete response
PR = partial response
SD = stable disease
PD = progressive disease
NE = not evaluable or treatment ended

Samples are prepared for IHC using standard procedures as described (Higgins, J P, et al., Am J. Clin Pathol., 112:241-7, 1999) using microwave heat-induced epitope retrieval in citrate buffer, and FKBP12 staining is detected using an anti-FKBP12 antibody (Affinity Bioreagents # PA1-026A). The signal is detected using SuperSignal West Pico Chemiluminescent Substrate (Pierce, Rockford, Ill.). Samples are assigned a score ranging from 0 to 3+, based on the level of staining as observed visually. Scores are assigned by three independent observers and the results averaged. The analyses are performed multiple times. In some analyses the scores are reduced to “positive” or “negative”, with 0 being considered negative and 1+, 2+, and 3+ being considered positive. In other analyses 0 and 1+ are considered negative with 2+ and 3+ being considered positive. In other analyses only 3+ is considered positive. Ten visual fields from different areas of each tumor are used for evaluation of the score. Negative control slides without primary antibody are included for each staining. Normal epithelium of the relevant tissue type or vascular endothelium known to express FKBP12 is used as a positive control.

Association between favorable response to therapy with AP 23573 and FKBP12 level is tested using chi-square and Fisher's tests. The scoring system that most accurately predicts favorable response is used for selecting patients for future trials.

### Example 5

### Use of Multiple Biomarkers to Evaluate Likelihood of Sensitivity to a Rapamycin Analog in Tumor Cell Lines

The experiments described in Example 2 are repeated except that in addition to assessing FKBP12 expression levels in the tumor cell lines, PTEN expression is also assessed. PTEN is detected by Western blot using PTEN-specific antibodies (A2B1) from Santa Cruz Biotechnology (Santa Cruz, Calif.) as described (Nagata, Y., et al, Cancer Cell, 6:117-127, 2004). Expression levels are quantitated as for FKBP12. Multivariate analysis is used to analyse the association between FKBP12 levels, PTEN levels, and sensitivity to AP23573. The data are analysed to determine whether use of FKBP12 and PTEN levels in conjunction is better able to evaluate the likelihood that a tumor cell line is sensitive to AP23573, e.g., to determine whether an increased FKBP12 level relative to a reference value, in conjunction with a decreased PTEN level relative to a reference value, is more strongly correlated with sensitivity to AP23573 than either biomarker alone.

### Example 6

### Use of Multiple Biomarkers to Evaluate Likelihood of Patient Response to a Rapamycin Analog

The experiments described in Example 4 are repeated except that in addition to assessing FKBP12 expression levels in the tumor samples, PTEN expression is also assessed using IHC. For analysis of PTEN, slides of formalin-fixed, paraffin-embedded tissue sections are incubated with PTEN antibody (Ab-2, 1:500) as described (Podsypanina et al, Proc. Natl. Acad. Sci. USA, 98:10320-10325, 2001). IHC is performed with the LSAB2 kit (DAKO, Carpentaria, Calif.), color development with 3-3′-diaminobenzidine, and counterstaining using hematoxylin. A semiquantitative scoring system based on staining intensity and distribution using the immunoreactive score (IRS) as described (Chui, et al, Br. J. Cancer, 73:1233-1236, 1996; Friedrichs, et al., Cancer 72:3641-3647, 1993) and as follows: IRS=SI (staining intensity)×PP (percentage of positive cells). SI is determined as 0=negative; 1=weak; 2=moderate; and 3=strong. PP is defined as 0, <1%; 1, 1%-10%; 2, 11%-50%; 3, 51%-80%; and 4, >80% positive cells. Ten visual fields from different areas of each tumor are used for evaluation of IRS. Negative control slides without primary antibody are included for each staining. Normal epithelium of the relevant tissue type or vascular endothelium known to express PTEN is used as a positive control.

Multivariate analysis is used to analyse the association between FKBP12 levels, PTEN levels, and sensitivity to AP23573. The data are analysed to determine whether use of FKBP12 and PTEN levels in conjunction is better able to evaluate the likelihood that a tumor cell line is sensitive to AP23573, e.g., to determine whether an increased FKBP12 level relative to a reference value, in conjunction with a decreased PTEN level relative to a reference value, is more strongly correlated with sensitivity to AP23573 than either biomarker alone.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

We claim:

1. A method for evaluating the likelihood that a tumor is sensitive to an mTOR inhibitor comprising steps of:
assessing the expression level or activity of an FKBP protein in a sample derived from a subject in need of such evaluation, wherein the subject has a tumor, or assessing an indicator of the expression or activity of the FKBP protein in the tumor in vivo, wherein the value of the indicator, relative to a reference value for the indicator, is predictive of the likelihood that the tumor is sensitive to the mTOR inhibitor.

2. The method of claim 1, wherein the mTOR inhibitor is rapamycin or a rapamycin analog.

3. The method of claim 1, wherein the rapamycin analog is selected from the group consisting of: temsirolimus (CCI-779), everolimus (RAD001; Certican), and AP23573.

4. The method of claim 1, wherein the FKBP protein is selected from the group consisting of: FKBP12, FKBP12.6, FKBP13, FKBP51, and FKBP 55.

5. The method of claim 1, wherein the FKBP is FKBP12.

6. The method of claim 1, wherein the FKBP is FKBP12.6.

7. The method of claim 1, wherein the sample comprises cells obtained from the subject.

8. The method of claim 7, wherein the cells are obtained from the tumor.

9. The method of claim 7, wherein the cells are isolated from the blood.

10. The method of claim 1, wherein the assessing comprises determining the expression level of a nucleic acid that encodes the FKBP protein.

11. The method of claim 1, wherein the assessing comprises measuring a binding activity of the FKBP protein.

12. The method of claim 11, wherein the binding activity is binding activity towards mTOR in the presence of rapamycin or a rapamycin analog.

13. The method of claim 1, wherein the binding activity is binding activity towards rapamycin or a rapamycin analog.

14. The method of claim 1, wherein the step of assessing comprises detecting a mutation that influences the expression level of the FKBP protein.

15. The method of claim 1, wherein the step of assessing comprises determining whether a gene that encodes the FKBP protein contains a mutation, and the reference value is a sequence for at least a portion of a normal gene that encodes the FKBP protein.

16. The method of claim 14, wherein presence of a mutation indicates a decreased likelihood that the tumor is sensitive to the mTOR inhibitor.

17. The method of claim 1, wherein the step of assessing comprises detecting the presence of a nucleic acid, or its complement, in the sample, wherein the nucleic acid encodes the FKBP protein.

18. The method of claim 17, wherein the nucleic acid is mRNA and the reference value was obtained from tumors that are not sensitive to an mTOR inhibitor, and wherein an increased level of the nucleic acid in the sample relative to the reference value indicates an increased likelihood that the tumor is sensitive to the mTOR inhibitor.

19. The method of claim 17, wherein the nucleic acid is mRNA and the reference value was obtained from normal cells or from tumors that are sensitive to an mTOR inhibitor, and wherein a decreased level of the nucleic acid in the sample relative to the reference value indicates a decreased likelihood that the tumor is sensitive to the mTOR inhibitor.

20. The method of claim 1, wherein the step of assessing comprises detecting the presence of the FKBP protein in the sample, determining the level of the FKBP protein in the sample, or both.

21. The method of claim 20, wherein the FKBP protein is FKBP12.

22. The method of claim 20, wherein the FKBP protein is FKBP12.6.

23. The method of claim 20, wherein the presence or level of the FKBP protein is detected using a reagent that specifically binds to the protein.

24. The method of claim 23, wherein the reagent is an antibody, antibody fragment, or antibody derivative.

25. The method of claim 20, wherein the FKBP protein is detected using immunofluorescence, flow cytometry, or immunohistochemistry.

26. The method of claim 23, wherein the reagent is a small molecule that is an FKBP ligand.

27. The method of claim 26, wherein the ligand is a labeled ligand.

28. The method of claim 26, wherein the FKBP protein is FKBP12, and the labeled ligand is AP1491.

29. The method of claim 20, wherein the reference value was obtained from tumors that are not sensitive to an mTOR inhibitor, and wherein an increased level of the FKBP protein in the sample relative to the reference value indicates an increased likelihood that the tumor is sensitive to the mTOR inhibitor.

30. The method of claim 20, wherein the reference value was obtained from normal cells or from tumors that are sensitive to an mTOR inhibitor, and wherein a decreased level of the FKBP protein in the sample relative to the reference value indicates a decreased likelihood that the tumor is sensitive to the mTOR inhibitor.

31. The method of claim 1, wherein the step of assessing comprises performing an imaging study.

32. The method of claim 31, wherein the imaging study is a functional imaging study.

33. The method of claim 1, wherein the tumor is selected from the group consisting of: sarcoma, prostate cancer, breast cancer, endometrial cancer, hematologic cancer, and brain cancer.

34. The method of claim 1, further comprising assessing at least one additional indicator of the likelihood that the tumor is sensitive to an mTOR inhibitor, wherein the value of the indicator, relative to a reference value for the indicator, is predictive of the likelihood that the tumor is sensitive to the mTOR inhibitor.

35. The method of claim 34, wherein the at least one additional indicator is selected from the group consisting of:
(a) the proportion or level of TSC2 protein that is phosphorylated;
(b) the proportion or level of AKT protein that is phosphorylated;
(c) the proportion or level of S6 protein that is phosphorylated;
(d) the proportion or level of S6 kinase protein that is phosphorylated;
(e) the proportion or level of 4E-BP1 protein that is phosphorylated;
(f) the proportion or level of mTOR protein that is phosphorylated;
(g) the level of cyclin D1 mRNA or protein, cyclin D3 mRNA or protein, myc mRNA or protein, or any combination of these;
(h) the level of HIF-1α mRNA or protein, HIF-2α mRNA or protein; HIF-1β mRNA or protein, or any combination of the foregoing;
(i) the level of VHL mRNA or protein or a mutation affecting VHL expression;
(j) the level of RHEB mRNA or protein or the activity of RHEB protein;
(k) the level of eIF4E mRNA or protein;
(l) the level of p27Kip1 mRNA or protein;
(m) the level of VEGF-R mRNA or protein;
(n) the level of IGF-1R mRNA or protein;
(o) the level of PTEN mRNA, protein, or activity, or presence of a mutation affecting PTEN expression or activity;
(p) the level of Raptor mRNA, protein, or activity, or presence of a mutation affecting Raptor expression or activity;
(q) the level of GβL mRNA, protein, or activity, or presence of a mutation affecting GβL expression or activity;
(r) the level of a circulating VEGF polypeptide; and
(s) the level of circulating endothelial cells (CECs), the level of circulating endothelial progenitor cells (CEPs), or both.

36. The method of claim 35, wherein one or more of (a)-(q) is measured in a sample derived from the tumor.

37. The method of claim 35, wherein a favorable value of two or more indicators is predictive of an increased likelihood that the tumor is sensitive to the mTOR inhibitor, relative to the likelihood predicted based on a favorable value of only one indicator.

38. A method for evaluating the likelihood that a tumor is sensitive to an mTOR inhibitor comprising steps of:
assessing at least two indicators as set forth in claim 35, wherein the values of the at least two indicators relative to reference values for those indicators are in combination predictive of the likelihood that the tumor is sensitive to the mTOR inhibitor, and wherein at least one of the indicators is an upstream indicator and at least one of the indicators is a downstream indicator.

39. A method for evaluating the likelihood that a tumor is sensitive to an mTOR inhibitor comprising steps of:
assessing at least two indicators as set forth in claim 35, wherein the values of the at least two indicators relative to reference values for those indicators in conjunction are predictive of the likelihood that the tumor is sensitive to the mTOR inhibitor, and wherein at least one of the indicators is an upstream indicator and at least one of the indicators is an angiogenesis-related indicator.

40. The method of claim 1, wherein the subject has not previously been treated with an mTOR inhibitor.

41. The method of claim 1, wherein the subject has previously received treatment for the tumor.

42. The method of claim 1, wherein the subject has been previously treated with a chemotherapeutic agent other than an mTOR inhibitor.

43. The method of claim 41, wherein the subject has been previously treated with a first mTOR inhibitor, and the method comprises evaluating the likelihood that the tumor sensitive to a second mTOR inhibitor.

44. A method of selecting a subject for treatment with an mTOR inhibitor comprising the steps of:
(a) evaluating the likelihood that the subject has a tumor that is sensitive to an mTOR inhibitor according to the method of claim 1; and
(b) selecting the subject as a suitable subject for initiating or continuing treatment with the mTOR inhibitor based on step (a).

45. A method for evaluating the likelihood that a tumor is sensitive to an mTOR inhibitor comprising steps of:
assessing an indicator of the likelihood that a tumor is sensitive to an mTOR inhibitor, wherein a subject suffering from the tumor has not been previously treated with an mTOR inhibitor;
administering an mTOR inhibitor to the subject; and
assessing the indicator again after a period of time, wherein an alteration in the indicator relative to a value for the indicator in a sample obtained prior to the administering step is predictive of the likelihood that the tumor is sensitive to the mTOR inhibitor, and wherein the indicator is selected from the group consisting of:
(a) the proportion or level of a phosphorylated form of a protein, wherein a decrease in the proportion or level of the phosphorylated form of a protein is indicative of an increased likelihood that the tumor is sensitive to the mTOR inhibitor relative to the likelihood if the proportion or level remains unchanged or increases, and wherein the protein is selected from the group consisting of: AKT, S6, S6 kinase, 4E-BP1, and TSC2;
(b) the level of a protein selected from the group consisting of: HIF-1α protein, HIF-2α protein, and VEGF, wherein a decrease in the level of the protein is indicative of an increased likelihood that the tumor is sensitive to the mTOR inhibitor, relative to the likelihood if the level remains unchanged or increases;
(c) the proportion or level of CECs, CEPs, or both; and
(d) the growth rate of the tumor as assessed by a functional imaging technique that assesses metabolic activity or DNA synthesis in the tumor.

46. The method of claim 45, further comprising the step of comparing an alteration in the indicator with a reference value for the alteration, wherein the reference value is derived from samples obtained from subjects who exhibited a favorable response to an mTOR inhibitor.

47. The method of claim 45, further comprising the step of comparing an alteration in the indicator with a reference value for the alteration, wherein the reference value is derived from samples obtained from subjects who did not exhibit a favorable response to an mTOR inhibitor.

48. The method of claim 47, wherein the alteration is a decrease in the proportion or level of the phosphorylated form of a protein of step (a) or a decrease in the level of a protein of step (b), and wherein if the alteration is larger than the reference value, the alteration is indicative of an increased likelihood that the tumor is to the mTOR inhibitor, relative to the likelihood if the alteration is not larger than the reference value.

49. A method for evaluating the likelihood that a tumor is sensitive to an mTOR inhibitor comprising steps of:
assessing an indicator selected from the group consisting of: (a) the level of myc mRNA or protein, or any combination of these; (b) the level of HIF-1α mRNA or protein, HIF-1β mRNA or protein, HIF-2α mRNA or protein or any combination of these; (c) the level of VHL mRNA or protein or presence of a mutation affecting VHL expression; (d) the level of RHEB mRNA or protein or the activity of RHEB protein or presence of a mutation affecting RHEB expression or activity; (e) the level of Raptor mRNA, protein, or activity, or presence of a mutation affecting Raptor expression or activity; and (f) the level of GβL mRNA, protein, or activity, or presence of a mutation affecting GβL expression or activity.

50. A method for identifying an indicator useful for predicting the likelihood that a tumor is sensitive to an mTOR inhibitor, the method comprising steps of:
(a) assessing an indicator of the expression or activity of a protein in each of a plurality of samples derived from subjects with tumors that exhibited a favorable response following administration of an mTOR inhibitor and arriving at a result, wherein the protein is selected from the group consisting of: an FKBP protein, HIF-1α protein, VHL protein, and RHEB protein;
(b) assessing an indicator of the expression or activity of the protein in each of a plurality of samples derived from subjects with tumors that did not exhibit a favorable response following administration of an mTOR inhibitor and arriving at a result; and
(c) identifying the indicator as useful for predicting the likelihood that a tumor is sensitive to the mTOR inhibitor if a significant difference exists between the result of steps (a) and (b).

51. The method of claim 50, wherein the protein is FKBP12 protein.

52. The method of claim 50, wherein the indicator is the expression level of the protein.

53. The method of claim 50, wherein the indicator is the presence of a mutation in a gene that encodes the protein.

54. The method of claim 50, wherein the indicator is the expression level of an mRNA that encodes the protein.

55. The method of claim 50, wherein the tumors are of a single tumor type.

56. The method of claim 55, wherein the tumor type is selected from the group consisting of: sarcoma, prostate cancer, breast cancer, endometrial cancer, hematologic cancer, and brain cancer.

57. The method of claim 50, wherein at least some of the samples are in a tissue microarray.

58. A method for evaluating the likelihood that a subject with a tumor will exhibit a favorable response to an mTOR inhibitor comprising steps of:
(a) performing a first functional imaging study of the tumor, wherein the first functional imaging study provides an indication of cell metabolism or cell proliferation in the tumor;
(b) administering an mTOR inhibitor to the subject following step (a);
(c) performing a second functional imaging study of the tumor, wherein the second functional imaging study provides an indication of cell metabolism or cell proliferation in the tumor, and wherein a detectable decrease in cell metabolism or cell proliferation in the tumor is indicative of an increased likelihood that the subject will experience a favorable response to the mTOR inhibitor.

59. A method of selecting a subject for treatment with an mTOR inhibitor comprising:
(a) obtaining first and second functional images of the tumor according to the method of claim 58;
(b) comparing results of the first and second functional imaging studies; and
(c) selecting the subject for treatment with the mTOR inhibitor if a detectable decrease in cell metabolism or cell proliferation is observed following administration of the mTOR inhibitor.

60. The method of claim 58, wherein at least one functional imaging study includes a PET scan.

61. The method of claim 58, wherein the first and second functional imaging studies include a PET scan.

62. A method for treating a subject suffering from a tumor, the method comprising the steps of:
evaluating the likelihood that the tumor is sensitive to an mTOR inhibitor according to the method of any of claims 1, 34, 35, 38, 39, 45 or 49; and
administering an mTOR inhibitor to the subject.

63. The method of claim 62, wherein the evaluating step indicates an increased likelihood that the tumor is sensitive to the mTOR inhibitor.

64. A method for treating a subject suffering from a tumor, the method comprising the steps of:
evaluating the likelihood that the subject will exhibit a favorable response to an mTOR inhibitor by a method that comprises evaluating the likelihood that the tumor is sensitive to an mTOR inhibitor according to the method of any claims 1, 34, 35, 38, 39, 45 or 49; and
administering an mTOR inhibitor to the subject.

65. The method of claim 64, wherein the evaluating step indicates an increased likelihood that the tumor is sensitive to the mTOR inhibitor.

66. A method of treating a subject with a tumor, the method comprising administering an mTOR inhibitor to the subject, wherein the likelihood that the tumor is sensitive to the mTOR inhibitor has been evaluated according to the method of any of claims 1, 34, 35, 38, 39, 45 or 49.

67. The method of claim 66, wherein said method indicated an increased likelihood that the subject will exhibit a favorable response to an mTOR inhibitor.

68. A kit for evaluating the likelihood that a tumor is sensitive to an mTOR inhibitor, the kit comprising a validated reagent for assessing the expression or activity of an FKBP protein in a sample obtained from a subject with a tumor, wherein a validated reagent is one that has been demonstrated to be of use in the method of claim 1.

69. The kit of claim 68, wherein the reagent is an antibody, antibody fragment, antibody derivative, or ligand that specifically binds to the FKBP protein.

70. The kit of claim 68, wherein the reagent is a nucleic acid that hybridizes to a nucleic acid that encodes the FKBP protein.

71. The kit of claim 68, wherein the FKBP protein is FKBP12.

72. The kit of claim 68, wherein the ligand is labeled.

73. The kit of claim 68, wherein the ligand is labeled FK506, labeled rapamycin, or a labeled rapamycin analog.

74. The kit of claim 68, wherein the ligand is AP1491.

75. The kit of claim 68, further comprising at least one item selected from the group consisting of: a reference sample, a positive control, a negative control, a substrate, an enzyme, a wash solution, a reagent for assessing a second indicator of the likelihood that the tumor is sensitive to an mTOR inhibitor; and instructions for use of the kit.

76. The kit of claim 75, wherein the reagent for assessing the second indicator of the likelihood that the tumor is sensitive to an mTOR inhibitor is an antibody, antibody fragment, antibody derivative, or ligand that specifically binds to a polypeptide selected from the group consisting of: PTEN, AKT, mTOR, TSC2, GβL, Raptor, S6, S6 kinase, eIF-4E, 4E-BP1, HIF-1α, and VHL.

77. The kit of claim 76, wherein the reagent specifically binds to a phosphorylated form of the polypeptide.

78. A kit for evaluating the likelihood that a tumor is sensitive to an mTOR inhibitor, the kit comprising a reagent for assessing an indicator of the expression or activity of an FKBP protein in a sample obtained from a subject with a tumor and further comprising at least one item selected from the group consisting of: a reagent for assessing a second indicator of the likelihood that the tumor is sensitive to an mTOR inhibitor, a reference sample, a positive control, a negative control, a substrate, an enzyme, and a wash solution.

79. A computer-readable medium on which is stored:
(a) a value for each of one or more indicators selected from the group consisting of: the level of expression or activity of an FKBP protein; the proportion or level of TSC2 protein that is phosphorylated; the proportion or level of AKT protein that is phosphorylated; the proportion or level of S6 protein that is phosphorylated; the proportion or level of S6 kinase protein that is phosphorylated; the proportion or level of 4E-BP1 protein that is phosphorylated; the proportion or level of mTOR protein that is phosphorylated; the level of cyclin D1 mRNA or protein, cyclin D3 mRNA or protein, myc mRNA or protein, or any combination of these; the level of HIF-1α mRNA or protein, HIF-2α mRNA or protein; HIF-1β mRNA or protein, or any combination of the foregoing; the level of VHL mRNA or protein or a mutation affecting VHL expression; the level of RHEB mRNA or protein or the activity of RHEB protein; the level of eIF4E mRNA or protein; the level of p27Kip1 mRNA or protein; the level of VEGF-R mRNA or protein; the level of IGF-1R mRNA or protein; the level of PTEN mRNA, protein, or activity, or a mutation affecting PTEN expression; the level of Raptor mRNA, protein, or activity, or a mutation affecting Raptor expression; the level of GβL mRNA, protein, or activity, or a mutation affecting GβL expression; the level of a circulating VEGF polypeptide; and the level of circulating endothelial cells (CECs), the level of circulating endothelial progenitor cells (CEPs), or both; and a value obtained from a functional imaging study, wherein the value for the one or more indicators was obtained from a tumor, from a sample derived from a tumor, or from a subject suffering from the tumor; and
(b) tumor-related information indicating (i) whether the tumor is sensitive or resistant to an mTOR inhibitor, (ii) whether a subject having the tumor exhibited a favorable or unfavorable response to an mTOR inhibitor, or (iii) both, wherein the value and the information are associated with one another.

80. The computer readable medium claim 79, wherein the indicator is the level of expression or activity of an FKBP protein.

81. The computer readable medium claim 79, wherein the indicator is the level of expression or activity of FKBP12 protein.

82. The computer readable medium claim 79, wherein the imaging study includes a PET scan.

83. The computer readable medium claim 79, wherein values for the indicator obtained prior to and following administration of an mTOR inhibitor to a subject are stored.

84. A computer readable medium on which is stored a plurality of values and tumor-related information for a plurality of tumors as set forth in claim 79, wherein each value is obtained from a tumor, from a sample derived from the tumor, or from a subject having the tumor, and wherein each value is associated with information related to the tumor.
